(19) United States
(12) Patent Application Publication
(10) Pub. No.: US 2018/0236109 A1
(43) Pub. Date: Aug. 23, 2018

(54) Pain Tracking by PET-imaging (Pain-TraP)

(71) Applicant: Universitat zu Koln, Koln (DE)

(72) Inventors: Tim HUCHO, Koln (DE); Bernd NEUMAIER, Koln (DE); Heike ENDEPOLS, Burscheid (DE); Achim SCHMIDTKO, Frankfurt am Main (DE)

(73) Assignee: Universitat zu Koln, Koln (DE)

(21) Appl. No.: 15/753,188

(22) PCT Filed: Aug. 19, 2016
(86) PCT No.: PCT/EP2016/069730

§ 371 Date: Feb. 16, 2018

(30) Foreign Application Priority Data
Aug. 20, 2015 (EP) 15181876.2

(51) Int. Cl. A61K 51/04; A61B 5/00; A61B 6/03; A61B 6/00

(52) U.S. Cl. CPC A61K 51/0455; A61B 5/4824; A61K 51/0402; A61B 6/50; A61B 6/037

(57) ABSTRACT

Subject matter of the present invention is PSMA Binding molecules for use in diagnosis and/or imaging of pain. Diagnosis or imaging of pain may be the visualization of the location of the origin of pain and/or the determination of the etiology of pain and/or the determination of the pain intensity and/or the stratification of subjects suffering from pain.

Subject matter of the present invention are PSMA-binding molecules for use in diagnosis and/or imaging of pain. Diagnosis or imaging of pain may be the visualization of the location of the origin of pain and/or the determination of the etiology of pain in subjects suffering from pain.

The gene folate hydrolase 1 (FOLH1) is coding for an enzyme with a number of different names. It is referred to in the scientific literature by the name of prostate specific membrane antigen (PSMA), N-acetylated-alpha-linked acidic dipeptidase (NAALADase) as well as by the name of glutamate carboxypeptidase II (GCPII). For simplicity reasons, we will use the name PSMA throughout the text.

PSMA is a zinc metalloenzyme which is known to locally increase the concentration of excitatory glutamate while decreasing the concentration of inhibitory NAAG. PSMA is a transmembrane protein with its enzymatic domain presented to the extracellular domain.

As determined by western blotting and immunocytochemistry, the enzyme is expressed in a number of tissues as reviewed recently in (Barinka et al., 2012). PSMA has been found in cells such as prostate (Troyer et al., 1995; Silver et al., 1997; Bostwick et al., 1998; Sokoloff et al., 2000; Mhawech-Fauceglia et al., 2007), nervous system (Berger et al., 1995; Sacha et al., 2007), kidney (Lopes et al., 1990; Silver et al., 1997; Chang et al., 1999; Mhawech-Fauceglia et al., 2007; Rovenska et al., 2008), and small intestine (Troyer et al., 1995; Silver et al., 1997; Sokoloff et al., 2000; Mhawech-Fauceglia et al., 2007; Rovenska et al., 2008). Beyond its expression in normal healthy humans, PSMA is highly upregulated in malignant tissues such as tumors derived from kidney, bladder, breast, colon and Schwann cells (Gala et al., 2000; Kinoshita et al., 2006; Mhawech-Fauceglia et al., 2007; Haffner et al., 2009; Wang et al., 2009) with highest concentrations reached in prostate cancer (Bostwick et al., 1998). This membrane bound enzyme shows hydrolytic activity of N-acetyl-aspartyl-glutamate (NAAG) (Robinson et al., 1987) and of folate (Pinto et al., 1996; Luthi-Carter et al., 1998). NAAG is produced by neurons while PSMA is mostly expressed by surrounding glia cells (Berger et al., 1995; Sacha et al., 2007). Released NAAG acts on metabotropic glutamate receptor 3, which is mostly alpha-i coupled and thus results in decrease of intracellular cAMP levels (Niswender and Conn, 2010). PSMA cleaves the peptide bond resulting in free glutamate Riveros and Orrego, 1984; Robinson et al., 1987; Baslow, 2000). Accordingly, the inhibitory input through mGluR3 is reduced while simultaneously the neuron-activating action of glutamate onto ionotropic glutamate receptors is increased (reviewed in (Doble, 1999; Lau and Tymianski, 2010)).

Glutamate regulation is central for neurobiology. There is a wide variety of neurobiological processes where glutamate is involved in (for review see (Lau and Tymianski, 2010). At the focus of research, glutamate is one of the central transmitters involved in neuronal synaptic transmission. Its ionotropic receptors are involved in acute depolarization as well as the long-term establishment of cellular changes by e.g. long-term potentiation. As a downside of glutamate action, excitotoxicity has been investigated in detail (reviewed in (Lau and Tymianski, 2010)). An overactivation of ionotropic glutamate receptors is believed to result in an excessive increase of intracellular calcium concentrations resulting in synapse/neurite retraction, neurodegeneration and apoptosis. This is believed to underlie e.g. secondary ischemic damage in CNS trauma.

As indicated by one of its names, Prostate Specific Membrane Antigen (PSMA) is intensively investigated in the context of cancer. Prostate cancer is among the most common cancers resulting in the death of about 30.000 men in 2014 in the USA (Marko et al., 2015). PSMA is strongly upregulated in prostate carcinoma cells (Akhtar 2013 1-6 [24]). In addition, it is highly expressed in neovascularization of nearly any solid tumor (Akhtar 2013 (8-11) [24]). Accordingly, PSMA binding compounds of various kinds have been developed and are under development for cancer diagnosis as well as for delivery of anti-cancer therapeutics (Marko et al., 2015; Srinivasarao et al., 2015).

However, PSMA is not only expressed in cancerous cells, but also among others along the nervous system (Berger et al. 1995; Sacha et al., 2007). The product of its activity, glutamate, is an excitatory transmitter present throughout the pain system (see review by (Wozniak et al., 2012) [27]). In the periphery, acute injections of agonists of glutamate receptors result in pain sensitization (Carlton et al., 1995; Jackson et al., 1995; Zhou et al., 1996; Davidson et al., 1997; Lawand et al., 1997; Carlton et al., 2001). Indeed, also pain inducing conditions result in local peripheral glutamate increases in animal models and patients (Omote et al., 1998; deGroot et al., 2000; McNearney et al., 2000). And conversely, inhibition of glutamate release or inhibitors of ionotropic glutamate receptors reduces pain in models of acute and chronic pain (Brown and Krupp, 2006; Coderre et al., 2007; Collins et al., 2010). Similarly, agonists of the inhibitory metabotropic glutamate receptors result also in pain reduction (Imre, 2007; Montana et al., 2009; Montana et al., 2011; Montana and Gereau, 2011; Zammataro et al., 2011). Whether or not the detection of the extracellular enzymatic domain of PSMA can be used in the imaging and/or diagnosis of pain, in particular in subjects suffering from pain, has not been investigated.

Pain is a huge individual and socioeconomic challenge. A large number of different diseases and syndromes are associated with chronic pain. Methods for diagnosis of pain are limited and do not result in clear mechanism-based insight into the aetiology of the individual pain (Fillingim et al., 2014). As pain is often present for many days to even weeks, months, and years, descriptions and quantification of changes in pain perception cannot be anything but purely subjective and of relative low quality. Methods of clinical pain diagnostic include questionnaires and recently also of methods such as “quantitative sensory testing”. Both depend again on the responses and self-reports of the patient, thus are highly subjective and often highly time consuming. The urgency to develop an objective measurement of pain becomes apparent with the problem, that it is often difficult to even define the exact location of the pain. Striking examples of such difficulties are for example the diffuse pain of multisegmental spinal cord degeneration leaving the clinician with the difficulty which dorsal ganglia segment should be targeted therapeutically. But also e.g. amputation pain does often not allow identifying the cause of the pain e.g. painful changes in the remaining stump of the respective extremity or pain-eliciting changes in the central nervous system. Thus, a method to identify the location of pain is urgently needed.

There is another shortcoming of the reliance of nearly all diagnostic approaches on the communication with the patient about the location, the presentation and the intensity of pain. Large groups of patients with special needs like children, dement elderly, mentally challenged, palliative, and/or intensive care patients can currently not being treated adequately due to the lack of reliable pain-self-reporting by the patients (Li et al., 2008; Barton et al., 2009; Herr et al., 2011; Greve et al., 2013). These patients are herein referred to as “patient(s) suspected to suffer from pain”. However, also in “normally” communicating patients, pain relief is achieved only after long periods of testing of various therapeutic options. This results in individual hardship, high medical costs, and huge societal costs due to e.g. loss of workforce. A method to visualize the origin of pain, to make the sensitivity of pain objectively measurable, and to give mechanistic insight into the aetiology of the respective pain is urgently needed.

One problem of current pain diagnostic is the nearly complete absence of diagnostic tools clearly identifying the underlying pain aetiology (Fillingim et al., 2014). Consequently, a mechanism based therapy cannot be initiated. This becomes especially apparent in the case of so called “chronic” (i.e. mostly longer than 3 months) pain patients. Usually one cannot even differentiate between changes in the afferent nociceptive system versus e.g. a psychological cause of pain. These two most contrasting pain mechanisms require completely opposite therapeutic approaches. An aetiology residing in the afferent nociceptive system could respond to classical pain therapeutics. Opioids, non-steroidal anti-inflammatory drugs (NSAIDs), and even more recently developed anti-convulsive drugs and anti-depressants (both of which are now known to also act on ion channels of the afferent nociceptive system) could result in significant alleviation of pain. However these classical drugs exhibit a number of side effects such as sedation, cognitive impairment, respiratory depression, tolerance, constipation, gastrointestinal bleeding, ulcers, myocardial infarction, stroke, ataxia, arrhythmias, nausea, fatigue, and addiction (Woodcock, 2009). Indeed, there are now more deaths by therapeutic opioids than by suicide and traffic accidents combined. Therefore, these drugs should only be prescribed if the chances of a therapeutic benefit are outweighing the side effects. A clearly detected pain-initiating alteration of the peripheral nociceptive nervous system would be such a situation potentially benefitting from these classical pain treatments. On the other hand, one cause underlying the perceived pain could be changes in the central nervous system. Especially, if this CNS-derived pain is due to changes of higher neuronal structures such as brain areas and/or psychological changes, these changes currently respond only marginally to classical pain therapeutics, if at all. Even worse, the negative side effects of the classical pharmacological pain drugs could aggravate the psychological condition rather than alleviating it. Accordingly, rather than titrating in classical pain therapeutic drugs and thereby further stressing the patient with negative side effects, these patients might rather gain from psychological and/or motivational training. However, currently these patients first require long-lasting attempts to find an effective analgesic drug. Indeed, the success of a therapeutic pharmacological treatment is currently part of the diagnostic process. Accordingly, the average time until diagnosis for painful situations not caused by classical alterations of the peripheral nociceptive system and/or alterations located in the spinal cord such as fibromyalgia is 12 years thereby producing a very long and deeply engraved pain history for the individual and huge cost for the society (Renfrey et al., 2003; Stewart et al., 2003; Andlin-Sobocki et al., 2005). Therefore, a diagnostic tool not only identifying the pain location but also differentially diagnosing aetiologies is urgently needed.

The present invention solves the above outlined problems by the provision of PSMA-binding molecules for use in diagnosis and/or imaging of pain, in particular in patients suffering from pain. The present invention provides also PSMA-binding molecules for use in diagnosis and/or imaging of pain in patients suspected to suffer from pain, but that show reduced or absence of ability to communicate.

According to one embodiment, the invention provides a PSMA-binding molecule comprising a detectable unit for use in the diagnosis and/or imaging of pain in a patient suffering from pain or in a patient that is suspected to suffer from pain.

According to one embodiment, the invention provides PSMA-binding molecule comprising a detectable unit for use in the diagnosis and/or imaging of pain, wherein said patient suspected to suffer from pain is reduced in its ability or unable to communicate verbally.

According to one embodiment, the invention provides a PSMA-binding molecule referred to in the preceding embodiment, wherein the detectable unit has a structure depicted in formula Compound I

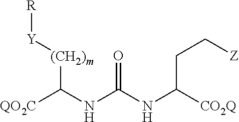

wherein
   Z is tetrazole or CO₂Q;
   each Q is hydrogen; and
wherein
   (A) m is 0, 1, 2, 3, 4, 5, or 6;
   R is a pyridine ring selected from the group consisting of

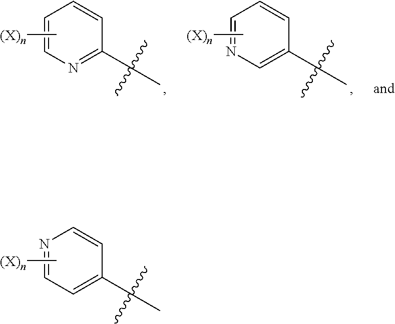

wherein X is a radioisotope of fluorine, a radioisotope of iodine, a radioisotope of bromine, a radioisotope of astatine, —NHN═CHR³, CH₂R³;
n is 1, 2, 3, 4, or 5;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)_{P}, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R³ is alkyl, alkenyl, alkynyl, aryl, or heteroaryl each of which is substituted by a radioisotope of fluorine, a radioisotope of iodine, a radioisotope of bromine, or a radioisotope of astatine.

According to one embodiment, the invention provides a PSMA-binding molecule for use in diagnosis and/or imaging of pain in a subject suffering from pain or in a patient that is suspected to suffer from pain according to any of the preceding embodiments, wherein Z is CO₂Q.

According to one embodiment, the invention provides a PSMA-binding molecule for use in diagnosis and/or imaging of pain in a subject suffering from pain or in a patient that is suspected to suffer from pain according to any of the preceding embodiments, wherein Q is hydrogen.

According to one embodiment, the invention provides a PSMA-binding molecule for use in diagnosis and/or imaging of pain in a subject suffering from pain or in a patient that is suspected to suffer from pain according to any one of the preceding embodiments, where m is 1, 2, 3, or 4.

According to one embodiment, the invention provides a PSMA-binding molecule for use in diagnosis and/or imaging of pain in a subject suffering from pain or in a patient that is suspected to suffer from pain according to any one of the preceding embodiments, having the structure

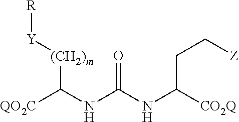

wherein
m is 0, 1, 2, 3, 4, 5, or 6;
R is a pyridine ring selected from the group consisting of

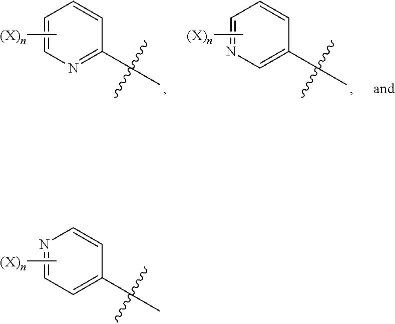

wherein X is a radioisotope of fluorine, a radioisotope of iodine, a radioisotope of bromine, a radioisotope of astatine, —NHN═CHR³;
each Q is independently selected from hydrogen or a protecting group;
   Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R is hydrogen, alkyl, aryl or heteroaryl, each of which may be substituted;
   Z is tetrazole or CO₂Q;
   R² is C₁-C₆ alkyl; and
   R³ is alkyl, alkenyl, alkynyl, aryl, or heteroaryl, each of which is substituted by fluorine, iodine, a radioisotope of fluorine, a radioisotope of iodine, chlorine, bromine, a radioisotope of bromine, or a radioisotope of astatine; NO₂, NH₂, N+(R²)₃, Sn(R²)₃, Si(R²)₃, Hg(R²), or B(OH)₂.

According to one embodiment, the invention provides a PSMA-binding molecule for use in diagnosis and/or imaging of pain in a subject suffering from pain or in a patient that is suspected to suffer from pain according to the preceding embodiments, having the structure

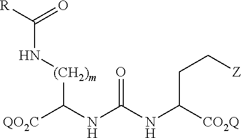

wherein m is not 0.

According to one embodiment, the invention provides a PSMA-binding molecule for use in diagnosis and/or imaging of pain in a subject suffering from pain or in a patient that is suspected to suffer from pain according to the preceding embodiments, where Z is CO₂Q, Q is hydrogen, and m is 4.

According to one embodiment, the invention provides a PSMA-binding molecule for use in diagnosis and/or imaging of pain in a subject suffering from pain or in a patient that is suspected to suffer from pain according to the preceding embodiments, having the structure

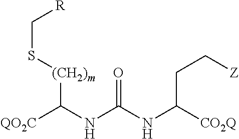

wherein m is not 0.

According to one embodiment, the invention provides a PSMA-binding molecule for use in diagnosis and/or imaging of pain in a subject suffering from pain or in a patient that is suspected to suffer from pain according to the preceding embodiments, where Z is CO₂Q, Q is hydrogen, and m is 1, 2, or 3.

According to one embodiment, the invention provides a PSMA-binding molecule for use in diagnosis and/or imaging of pain in a subject suffering from pain or in a patient that is suspected to suffer from pain according to the preceding, wherein m is 0, 1, 2, 3, 4, 5, or 6;
Y is O, S, N(R′), C(O), NR1C(O), C(O)N(R′), OC(O), C(O)O, NR′C(O)NR′, NR′C(S)NR, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R is

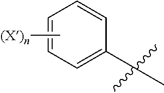

wherein
   X1 is selected from the group consisting of NHNH₂, —NHN═CHR³, —NHNH—CH₂R³; wherein R³ is alkyl, alkenyl, alkynyl, aryl, or heteroaryl, each of which is substituted by fluorine, iodine, a radioisotope of fluorine, a radioisotope of iodine, bromine, a radioisotope of bromine, or a radioisotope of astatine; NO₂, NH₂, N+(R²)₃, Sn(R²)₃, Si(R²)₃, Hg(R²), and B(OH)₂, where R² is C₁-C₆ alkyl; n is 1, 2, 3, 4, or 5.

According to one embodiment, the invention provides a PSMA-binding molecule for use in diagnosis and/or imaging of pain in a subject suffering from pain or in a patient that is suspected to suffer from pain according to the preceding embodiments, wherein n is 1.

According to one embodiment, the invention provides a PSMA-binding molecule for use in diagnosis and/or imaging of pain in a subject suffering from pain or in a patient that is suspected to suffer from pain according to the preceding embodiments, wherein X or X′ is fluorine, iodine, or a radioisotope of fluorine or iodine, bromine, a radioisotope of bromine, or a radioisotope of astatine.

According to one embodiment, the invention provides a PSMA-binding molecule for use in diagnosis and/or imaging of pain in a subject suffering from pain or in a patient that is suspected to suffer from pain according to the preceding embodiments, wherein X or X′ is fluorine, iodine, or a radioisotope of fluorine or iodine.

According to one embodiment, the invention provides a PSMA-binding molecule for use in diagnosis and/or imaging of pain in a subject suffering from pain or in a patient that is suspected to suffer from pain according to the preceding embodiments, wherein m is 4, Y is NR′, and R is

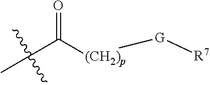

wherein G is O, NR′ or a covalent bond
   p is 1, 2, 3, or 4, and
   R⁷ is selected from the group consisting of NH₂, N═CHR³, NH—CH₂R³, wherein R³ is alkyl, alkenyl, alkynyl, aryl, heteroaryl each of which is substituted by fluorine, iodine, a radioisotope of fluorine, a radioisotope of iodine, chlorine bromine, a radioisotope of bromine, or a radioisotope of astatine NO₂, NH₂, N+(R₂)₃, Sn(R²)₃, Si(R²)₃, Hg(R²), and B(OH)₂, wherein R² is C₁-C₆ alkyl.

According to one embodiment, the invention provides a PSMA-binding molecule for use in diagnosis and/or imaging of pain in a subject suffering from pain or in a patient that is suspected to suffer from pain according to the preceding embodiments, wherein G is O or NR′.

According to one embodiment, the invention provides a PSMA-binding molecule for use in diagnosis and/or imaging of pain in a subject suffering from pain or in a patient that is suspected to suffer from pain according to the preceding embodiments, wherein R comprises a radioisotope.

According to one embodiment, the invention provides a PSMA-binding molecule for use in diagnosis and/or imaging of pain in a subject suffering from pain or in a patient that is suspected to suffer from pain according to the preceding embodiments, wherein the radioisotope is selected from the group consisting of ¹⁸F, ⁶⁸Ga, ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br and ²¹¹At.

According to one embodiment, the invention provides a PSMA-binding molecule for use in diagnosis and/or imaging of pain in a subject suffering from pain or in a patient that is suspected to suffer from pain according to the preceding embodiments selected from the group consisting of

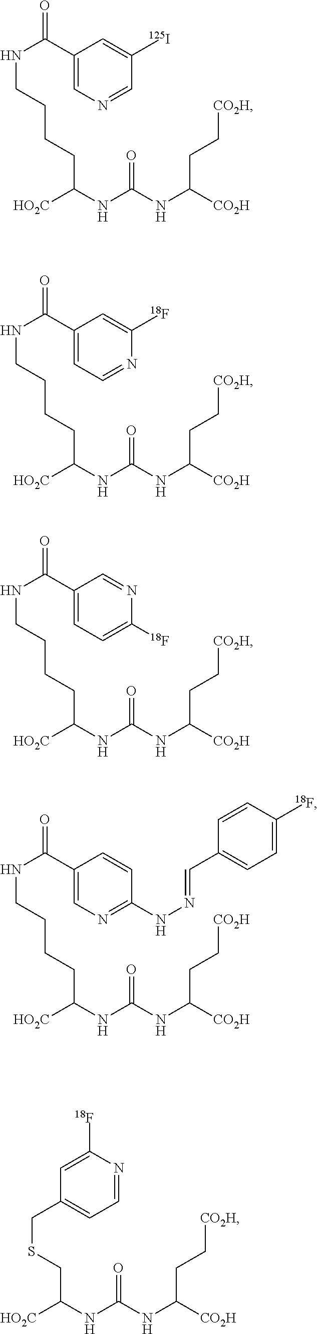

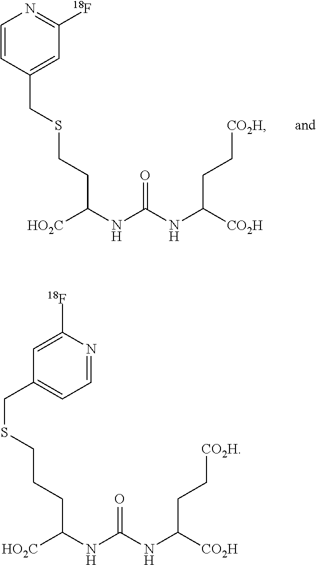 [SMILES: O=C(O)CCC(NC(=O)NC(CCSCc1ccnc([18F])c1)C(=O)O)C(=O)O ; O=C(O)CCC(NC(=O)NC(CCCSCc1ccnc([18F])c1)C(=O)O)C(=O)O]

According to one embodiment, the invention provides a PSMA-binding molecule for use in diagnosis and/or imaging of pain in a subject suffering from pain or in a patient that is suspected to suffer from pain according to the preceding embodiments having the structure

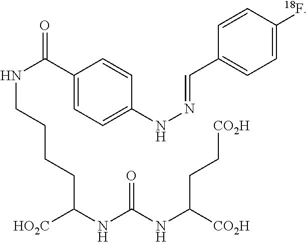

According to one embodiment, the invention provides a PSMA-binding molecule for use in diagnosis and/or imaging of pain in a subject suffering from pain or in a patient that is suspected to suffer from pain according to the preceding embodiments having the structure

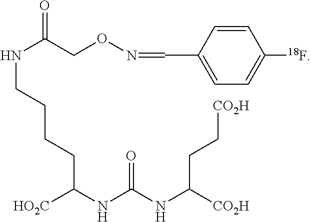

According to further embodiments of the invention, the PSMA-binding molecule as defined in any of the preceding embodiments is for use in diagnosis or imaging of pain, wherein the pain eliciting location is visualized, or it is for use in a method of diagnosis or imaging of pain, wherein the pain eliciting location is visualized.

According to one embodiment, the invention provides a PSMA-binding molecule for use in diagnosis and/or imaging of pain in a subject suffering from pain or in a patient that is suspected to suffer from pain according to the preceding embodiments the PSMA-binding molecule as defined in any of the preceding embodiments, wherein the level of enzyme PSMA is increased at a site of pain along a peripheral nerve or parts thereof.

According to one embodiment, the invention provides a PSMA-binding molecule for use in diagnosis and/or imaging of pain in a subject suffering from pain or in a patient that is suspected to suffer from pain according to the preceding embodiments, wherein the increased level of enzyme PSMA at said site of pain is detected as intensity of said PSMA-binding molecule comprising a detectable unit (hereinafter also referred to as “tracer”) after administration to said subject and wherein said tracer compound intensity at the site of pain is statistically increased in comparison to a) said tracer compound intensity at the site of an unaffected contralateral site and/or b) to a threshold that has been statistically determined.

According to one embodiment, the invention provides a PSMA-binding molecule for use in diagnosis and/or imaging of pain in a subject suffering from pain or in a patient that is suspected to suffer from pain according to the preceding embodiments, wherein diagnosis or imaging of pain may be the visualization of the pain eliciting location, the determination of pain sensitivity, and/or the determination of the aetiology of pain.

According to one embodiment, the invention provides a PSMA-binding molecule for use in diagnosis and/or imaging of pain in a subject suffering from pain or in a patient that is suspected to suffer from pain according to the preceding embodiments, wherein it is differentiated between peripherally caused pain (peripheral pain) versus central and periphery independent pain.

According to one embodiment, the invention provides a PSMA-binding molecule for use in diagnosis and/or imaging of pain in a subject suffering from pain or in a patient that is suspected to suffer from pain according to the preceding embodiments, wherein it is determined whether said subject suffers from inflammatory pain or neuropathic pain.

According to another embodiment of the invention, the PSMA-binding molecule according to any one of the preceding embodiments is for use in the manufacture of a kit for the diagnosis and/or imaging of pain in a patient suffering from pain according or in a patient that is suspected to suffer from pain to any of the preceding claims.

According to another embodiment of the invention, a kit comprising a container comprising PSMA-binding molecule as defined in any one of the preceding embodiments for the diagnosis and/or imaging of pain, optionally comprising instructions for use, and further optionally comprising information on the interpretation of imaging results is provided.

According to another embodiment of the invention, a method for diagnosing or imaging of pain in a subject suffering from pain or in a patient that is suspected to suffer from pain comprising administering to said subject an effective amount of a compound according to any of the preceding embodiments is provided.

According to another embodiment of the invention, an in vitro method of imaging cells, organs, tissue samples is provided, wherein the cells, organs or tissue samples are exposed to a chemical or physical stimulus suspect to be involved in the development or reduction of pain, and the expression and/or quantity of PSMA is determined using a PSMA-binding molecule as defined in any one of the preceding embodiments.

### DETAILED DESCRIPTION

As used herein, “pain” is defined according to the International Association for the Study of Pain (IASP), i.e. that pain is an unpleasant sensory and emotional experience associated with actual or potential tissue damage, or described in terms of such damage (Cortelli et al., 2013). This higher brain experience is evoked by neuronal activity of neurons of the peripheral and/or central nervous system involved in pain, the so called nociceptive nervous system.

In the context of this invention “pain” is defined as “pain detectable by a PSMA-binding molecule according to the invention. “Pain detectable by PSMA-binding molecule according to the invention” is defined by the involvement of the peripheral nociceptive system. It is further defined by an increase of the signal derived from the detectable unit of the PSMA-binding molecule according to the invention along the nerve as detected by a PET-scanner.

“Increase of the signal derived from the detectable unit of the PSMA-binding molecule according to the invention” is defined as the statistically relevant increase over a reference.

“Statistically relevant increase” is defined as a less than 5% probability of erroneously interpreting a coincidental difference between two similar measurements as a “real” difference. This so-called significance threshold (p<0.05) is the most important statistical parameter used to judge in biological experiments if a result is to be interpreted as “effect” or “no effect”. Depending on circumstances, the error probability (p-value) may be calculated with different statistical tests such as 1.) Students t-test if two groups of subjects are compared (e.g. patients with healthy persons). 2.) Paired t-test if one side of the body is compared to the other. 3.) Repeated measures ANOVA if measurements at different time points in the same subjects are compared and/or any other suitable statistical test for the evaluation of the difference between groups of measurements. Furthermore, to determine if a statistically significant difference is big enough to be meaningful, the effect size is calculated. The effect size is defined as the average difference divided by the variance of measurements—that means it is standardized to the inherent variability of the measured variable. There are custom thresholds defining low, middle and high effect sizes.

“Reference” can be of two kinds: In pain being suspected or being truly occurring only on one side of the body, the measurements of a collection of the same structure on the contralateral side can be used as reference to determine a normal signal derived from the previously applied PSMA-binding molecule with detectable unit according to the invention.

Alternatively, in pain being suspected or being truly occurring on both sides of the body in the same structures (e.g. both hands) such contralateral values cannot be used as reference.

Instead, such reference values need to be taken by measurements of the same structure as in the patient in “healthy” individuals (i.e. those not suffering from pain). Obtained data can be compared by measuring the affected side and comparing it with the average of unaffected sides.

As used herein pain may be “inflammatory pain” or “neuropathic pain”.

As used herein “inflammatory pain” is elicited by inflammatory changes in the surrounding of nociceptive neurons. These changes are accompanied by changes in the intercellular space by secretion of inflammatory mediators such as cytokines by changes in the local pH, and by others. These changes in turn result in activation of the nociceptive nerve and/or in sensitization to mechanical/thermal/chemical stimuli thus lowering the activation threshold and thereby resulting in increased nociceptive neuron activity.

As used herein “neuropathic pain” means that, the surrounding of the nerve is not the direct reason for the painfully increased or overactivity of the nociceptive nerve. Instead, the nerve is changed. This results in sensitization to mechanical/thermal/chemical stimuli or in spontaneous depolarizations of the membrane potential thereby resulting in enhanced nociceptive activity. Among neuropathic types of pain one may differentiate from central neuropathic, where in the former the functionality of the peripheral nociceptive neuron has changed, while in the latter the functionality of the central nociceptive neuron has changed. In preferred embodiments of the invention, peripheral neuropathic pain is diagnosed or imaged. When a positive PSMA signal is obtained in the periphery, it is assumed that the pain has its cause also in the periphery. Further, when a patient indicates that he is suffering from pain, but no peripheral positive PSMA signal is obtained, it can be assumed that the source or caused of pain is not peripheral, but central, or that the source is not the bodily region subjected to an imaging method. Respective methods are subject to the present invention.

As used herein, the “Visualization of pain eliciting location” is defined as the increase of the increase of the signal derived from the PSMA-binding molecule according to the present invention in comparison to a reference site. If there is an increase, this defines the peripheral pain eliciting location. As used herein, we are able to differentiate two major different mechanisms of pain-initiation: Peripheral inflammatory pain presents itself in our method as a local increase of the tracer signal (i.e. the signal derived from the detectable unit of the PSMA-binding molecule) at one or multiple sites while the tracer signal along the nerve-plexus connecting the peripheral site of signal-increase with the spinal cord does not show an increased PSMA-binging molecule's signal. In contrast, peripheral neuropathic pain presents itself as an increase at a potential site of lesion with in addition also an increase of the tracer signal along the nerve plexus connecting the site of lesion with the spinal cord.

As used herein, “pain sensitivity” means the activation threshold to a given stimulus (e.g. pressure, temperature, chemical) of peripheral nociceptive neurons which leads to the activation of the so called primary nociceptive neuron in the periphery resulting in the activation of the secondary nociceptive neurons in the spinal cord ultimately eliciting pain in the CNS. The activation threshold defines the sensitivity of the individual nociceptive neuron. This activation threshold can be altered by various factors. Accordingly, the individual nerve and thereby the respective individual can be of varying sensitivity toward pain eliciting stimuli. As a consequence, commonly sensitization i.e. lowering of the activation threshold results in the experience of more pain as more stimuli exceed the respective threshold. Sensitization can be so strong that even the normal environment of the nerve with its pressure, temperature and/or chemical properties can result in activation of the nociceptive neurons resulting often in spontaneous pain. Therefore, it is of high importance to determine the pain sensitivity of an individual.

As mentioned above, the present invention relates to the PSMA-binding molecules for use in diagnosis of pain according to any of the preceding embodiments, wherein it is differentiated between peripherally caused pain versus central and periphery independent pain. Together with the patients self-reporting about his/her pain state the visualization of the pain eliciting location may allow to define peripherally elicited pain versus periphery independent, i.e. central pain. If the patient is in pain but no peripheral pain eliciting location is detectable, then the pain eliciting site may be in the central nervous system.

As used herein, patients suffering from pain may be those presenting themselves at the physician with complaints of pain of any origin, e.g. inflammatory pain, pain due to autoimmune diseases (e.g. rheumatoid arthritis, etc.), pain from accidents, wounds, infections, broken bones, swellings, pain in limbs or any other part of the body, etc.

As used herein, patients suspected to suffer from pain are those that are unable to communicate with the treating physician, medical staff, relatives, etc., but which present visible signs, physiological reactions or a behavior suggesting pain. Visible signs are, for example, wounds, swellings, erythema, bruises, visible signs of infection, e.g. exudates, purulence, or signs obtained using imaging or palpation methods, with MRI, X-ray, ultrasonic analysis, PET, e.g. ischemia, broken bones; visible signs are also facial expressions suggesting pain and defensive behavior upon manipulation/touching of potentially affected bodily areas; signs of sympathomimetic activation, e.g. tachycardia, high blood pressure, dilated pupils, sweating; tissue alterations in regions that are sensitively innervated, etc. Further, pain can be suspected in patients that are unable to communicate and who have been exposed to, or suspected to have been exposed to, strikes, pushing, pulling, shaking, beating, stitches, and burns, entry or absorption of solid material into the body, exposure to heat or cold, acids, and/or bases, exposure to drugs, e.g. narcotics, alcohol, synthetic amphetamines, etc. Such patients may for example be children, dement elderly, mentally challenged, palliative, and/or intensive care patients.

As used herein, a PSMA-binding molecule designates any molecule that binds to PSMA and has a detectable unit, wherein said detectable unit may be identified using imaging methods, preferably PET, SPECT, MR, and OI.

As used herein, a PSMA-binding molecule comprises biological molecules and small molecules as long as they can be labeled with a detectable substance, e.g. a radionuclide. Biological molecules comprise antibodies and fragments or derivatives thereof.

In preferred embodiments, the detectable units of PSMA-binding molecules are parts of small molecules, e.g. those of compounds according to formula (I).

Therefore, embodiments of the invention include compounds according to formula I, shown below:

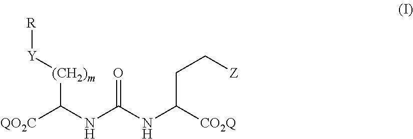

wherein Z is tetrazole or CO₂Q, and each Q is hydrogen.

In exemplary embodiments, m is 0, 1, 2, 3, 4, 5, or 6, R is a pyridine ring selected from the group consisting of

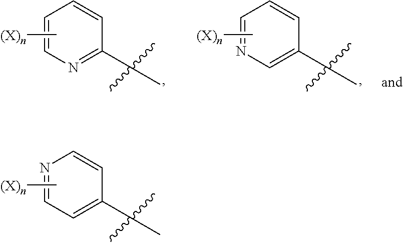

wherein X is a radioisotope of fluorine, a radioisotope of iodine, a radioisotope of bromine, a radioisotope of astatine, NHN═CHR³; n is 1, 2, 3, 4, or 5; and R³ is alkyl, alkenyl, alkynyl, aryl, or heteroaryl each of which is substituted by a radioisotope of fluorine, a radioisotope of iodine, a radioisotope of bromine, or a radioisotope of astatine; or a pharmaceutically acceptable salt thereof.

In exemplary embodiments of the above formula (I)

Z is tetrazole or CO₂Q
m is 0, 1, 2, 3, 4, 5, or 6, R is a pyridine ring selected from the group consisting of wherein X is fluorine, iodine, a radioisotope of fluorine, a radioisotope of iodine, chlorine, bromine, a radioisotope of bromine, a radioisotope of astatine, NO₂, NH₂, N⁺(R²)₃, NHNH₂, —NHN═CHR³, —NHNH—CH₂R³; n is 1, 2, 3, 4, or 5; Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; p is 1, 2, or 3, R′ is H or C₁-C₆ alkyl, and R⁸ is alkyl, aryl or heteroaryl, each of which may be substituted; R² is C₁-C₆ alkyl; and R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine, NO₂, NH₂, N+(R²)₃, or a pharmaceutically acceptable salt thereof.

In other embodiments, m is 0, 1, 2, 3, 4, 5, or 6; Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; p is 1, 2, or 3; R′ is H or C₁-C₆ alkyl; R⁸ is alkyl, aryl or heteroaryl, each of which may be substituted; R is

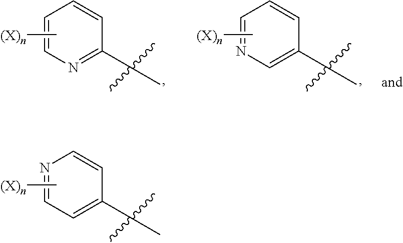

wherein X′ is selected from the group consisting of NHNH₂, —NHN═CHR³, and —NHNH—CH₂R³; wherein R³ is alkyl, alkenyl, alkynyl, aryl, or heteroaryl each of which is substituted by fluorine, iodine, a radioisotope of fluorine, a radioisotope of iodine, chlorine, bromine, a radioisotope of bromine, or a radioisotope of astatine; NO₂, NH₂, N⁺(R²)₃; R² is C₁-C₆ alkyl; n is 1, 2, 3, 4, or 5; or a pharmaceutically acceptable salt thereof.

In yet other embodiments (C), m is 4; Y is NR′; and R is

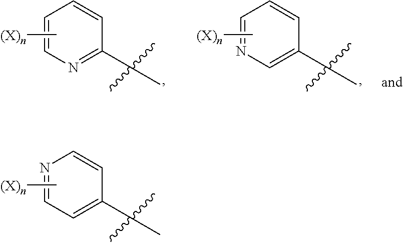

wherein G is O, NR′ or a covalent bond; R′ is H or C₁-C₆ alkyl; p is 1, 2, 3, or 4, and R⁷ is selected from the group consisting of NH₂, N═CHR³, NH—CH₂R³, wherein R³ is alkyl, alkenyl, alkynyl, aryl, or heteroaryl each of which is substituted by fluorine, iodine, a radioisotope of fluorine, a radioisotope of iodine, bromine, a radioisotope of bromine, or a radioisotope of astatine; NO₂, NH₂, N⁺(R²)₃; R² is C₁-C₆ alkyl; or a pharmaceutically acceptable salt thereof.

In some embodiments, R⁸ is alkyl, aryl or heteroaryl, each of which may be substituted. In certain embodiments, R⁸ describes the sidechain of a natural or synthetic α-amino acid. Specific examples of R⁸ include hydrogen, methyl (CH₃), isopropyl (CH(CH₃)₂), 2,2-dimethylethyl (CH₂CH(CH₃)₂), 2-methylpropyl (CH(CH₃)CH₂CH₃), phenyl, 4-hydroxyphenyl, hydroxymethyl (CH₂OH), carboxymethyl (CH₂CO₂H), thiomethyl (CH₂SH), imidazolylmethyl, indolylmethyl, and so forth.

In another aspect, the invention provides a compound of formula II:

A-(B)_{b}—C (II);

wherein A is a metal chelator; suitable chelators consist of but not limited to DOTA, NOTA, DTPA, cDTPA, CHX-A″-DTPA, TETA, NODAGA, HBED, DFO, DOTAGA; PCTA, MA-NOTMP; TRAP-Pr, NOPO; DOTPI, H₄OCTAPA; DOTAGA; LI-1,2HOPO; H₂dedPA, AAZTA, DATA^{x}; B is a linker; C is a PSMA-binding molecule; and b is 1-5.

In certain embodiments, the invention provides a compound of formula III:

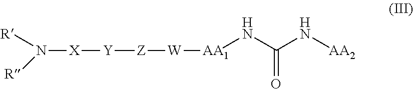

wherein
R′ is —CO—NR^{x}R^{y}—, —CS^{x}R^{y}—, COR^{x}, CSR^{x}, C(NR^{x})R^{x}, —S(O)ₚR^{x}—, —CO₂—NR^{x}R^{y}—, or optionally substituted alkyl;
R^{x} is optionally substituted aryl or optionally substituted alkyl;
R^{y} is H, optionally substituted aryl or optionally substituted alkyl;
X and Z are each independently C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, or a bond, each of which may be substituted with 0-5 R_{A};
Y and W are each independently —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl.

In one embodiment, AA₁ and AA₂ are each independently a natural amino acid. In a further embodiment, AA₁ and AA₂ are each independently lysine, glutamic acid, tyrosine, or cysteine.

In another embodiment, R′ is —CO—NR^{x}R^{y}, —CS—NR^{x}R^{y}, COR^{x}, CSR^{x}, or optionally substituted alkyl.

In still another embodiment, X is C₁-C₈ alkyl, C₁-C₈ alkoxy, or a bond, which may be substituted with 0-5 R_{A}; and R_{A} for each occurrence, is halogen, hydroxy, amino, cyano, nitro, or CO₂H.

In certain embodiments, Z is C₁-C₈ alkyl, C₁-C₈ alkoxy, or a bond, which may be substituted with 0-5 R_{A}; and R_{A} for each occurrence, is halogen, hydroxy, amino, cyano, nitro, or CO₂H.

In yet another embodiment, Y is —O—, —NH—, —NR_{B}—, —NH—CO—, —NH—CO₂—, —NR_{B}—CO—, —NR_{B}—CO₂—; —CO—NH—, —CO₂—NH—, —CO—NR_{B}—, or —CO₂—NR_{B}—. In a further embodiment, Y is —O—, —NH—CO— or —NR_{B}—CO—.

In other embodiments, the invention provides a compound of formula IV:

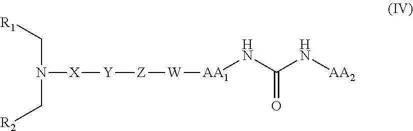

wherein
R₁ and R₂ are each independently selected from optionally substituted aryl, optionally substituted heteroaryl, optionally substituted heterocyclo, —COOH, hydroxyl, optionally substituted alkoxy, amino, optionally substituted mono or dialkylamino, thiol, and optionally substituted alkylthiol;
AA₁ and AA₂ are each independently a natural or unnatural amino acid;
X and Z are each independently C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₅ alkoxy, or a bond, each of which may be substituted with 0-5 R_{A};
Y is —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl.

In a further embodiment, AA₁ and AA₂ are each independently a natural amino acid. In still another further embodiment, AA₁ and AA₂ are each independently lysine, glutamic acid, tyrosine, or cysteine.

In certain embodiments, R₁ is phenyl, 1-naphthyl, 2-naphthyl, pyridyl, pyrimidinyl, pyrazinyl, pyridizinyl, quinolinyl, thienyl, thiazolyl, oxazolyl, isoxazolyl, pyrrolyl, furanyl, isoquinolinyl, imiazolyl, or triazolyl, each of which is optionally mono-, di-, or tri-substituted with R_{C}; or R₁ is —COOH, hydroxyl, alkoxy, amino, mono or dialkylamino, and R_{C} is halogen, hydroxy, amino, cyano, nitro, CO₂H, alkyl, alkoxy, mono or dialkylamino, aryl, or heteroaryl.

In another embodiment, R₂ is phenyl, 1-naphthyl, 2-naphthyl, pyridyl, pyrimidinyl, pyrazinyl, pyridizinyl, quinolinyl, thienyl, thiazolyl, oxazolyl, isoxazolyl, pyrrolyl, furanyl, isoquinolinyl, or triazolyl, each of which is optionally mono-, di-, or tri-substituted with R_{C}; or R₂ is —COOH, hydroxyl, alkoxy, amino, mono or dialkylamino, and R_{C} is halogen, hydroxy, amino, cyano, nitro, CO₂H, alkyl, alkoxy, mono or dialkylamino, aryl, or heteroaryl.

In one embodiment, X is C₁-C₈ alkyl, C₁-C₈ alkoxy, or a bond, which may be substituted with 0-5 R_{A}; and R_{A} for each occurrence, is halogen, hydroxy, amino, cyano, nitro, or CO₂H.

In another embodiment, Z is C₁-C₈ alkyl, C₁-C₈ alkoxy, or a bond, which may be substituted with 0-5 R_{A}; and R_{A} for each occurrence, is halogen, hydroxy, amino, cyano, nitro, or CO₂H.

In still another embodiment, Y is —O—, —NH—, —NR_{B}—, —NH—CO—, —NH—CO₂—, —NR_{B}—CO—, —NR_{B}—CO₂—; —CO—NH—, —CO₂—NH—, —CO—NR_{B}—, or —CO₂—NR_{B}—; in certain instances, Y is —O—, —NH—CO— or —NR_{B}—CO—.

In certain embodiments, the invention provides a compound of formula V:

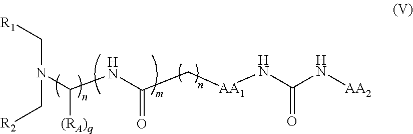

wherein
AA₁ and AA₂ are each independently a natural amino acid;
R₁ is pyridyl, pyrimidinyl, pyrazinyl, pyridizinyl, quinolinyl, thienyl, thiazolyl, oxazolyl, isoxazolyl, pyrrolyl, furanyl, isoquinolinyl, imiazolyl, or triazolyl;
R₂ is pyridyl, pyrimidinyl, pyrazinyl, pyridizinyl, quinolinyl, thienyl, thiazolyl, oxazolyl, isoxazolyl, pyrrolyl, furanyl, isoquinolinyl, or triazolyl, —COOH, hydroxyl, alkoxy, amino, mono or dialkylamino;
R_{A}, for each occurrence, is halogen, hydroxy, amino, cyano, nitro, or CO₂H;
m is 0 or 1;
each n is independently 1-8; and
each q is independently 0 or 1.

In one embodiment, AA₁ is lysine and AA₂ is glutamic acid or tyrosine. In a further embodiment, AA₁ is lysine and AA₂ is cysteine or tyrosine.

In certain embodiments, each n is independently 5-7. In other embodiments, m is 1.

In one embodiment, the invention provides for a compound of formula VI:

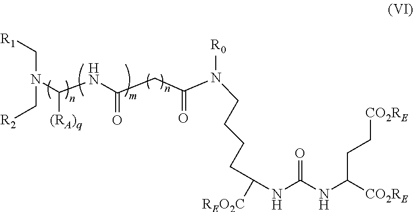

wherein
each R_{D} is independently H, optionally substituted alkyl, optionally substituted cycloalkyl, optionally substituted aryl, optionally substituted heteroaryl, optionally substituted heterocyclo, or optionally substituted aralkyl;
each R_{E} is independently H, optionally substituted alkyl, optionally substituted cycloalkyl, optionally substituted aryl, optionally substituted heteroaryl, optionally substituted heterocyclo, or optionally substituted aralkyl;
R₁ is pyridyl, pyrimidinyl, pyrazinyl, pyridizinyl, isoquinolinyl, imiazolyl, or quinolinyl;
R₂ is pyridyl, pyrimidinyl, pyrazinyl, pyridizinyl, isoquinolinyl, quinolinyl; —COOH, hydroxyl, alkoxy, amino, mono or dialkylamino;
R_{A}, for each occurrence, is hydroxy, amino, or CO₂H;
each m is independently 0 or 1; and
each n is independently 1-8.

In certain embodiments, R₁ is pyridyl, isoquinolinyl, imiazolyl, or quinolinyl. In other embodiments, R₂ is pyridyl, isoquinolinyl, quinolinyl, or —COOH.

In still another embodiment, each n is independently 5-7. In yet another embodiment, m is 1.

In certain embodiments, the invention provides a compound selected from the following:

In another embodiment, the invention provides a compound of formula VII:

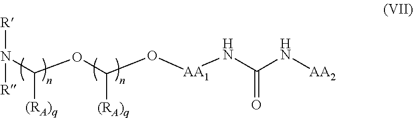

wherein
AA₁ and AA₂ are each independently a natural amino acid;
R′ is —CO—NR^{x}R^{y}—, —CS—NR^{x}R^{y}, COR^{x}, CSR^{x}, C(NR^{x})R^{x}, —S(O)ₚR^{x}, —CO₂—NR^{x}R^{y}, or optionally substituted alkyl;
R″ is H or optionally substituted alkyl;
R^{x} is optionally substituted aryl or optionally substituted alkyl;
R′ is H, optionally substituted aryl or optionally substituted alkyl;
R_{A}, for each occurrence, is halogen, hydroxy, amino, cyano, nitro, or CO₂H;
each n is independently 0-8; and
each q is independently 0 or 1.

In another embodiment, the invention provides a compound of formula VIII:

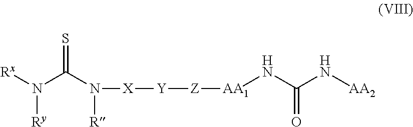

wherein
R″ is H or optionally substituted alkyl;
R^{x} is optionally substituted aryl or optionally substituted alkyl;
R^{y} is H, optionally substituted aryl or optionally substituted alkyl;
AA₁ and AA₂ are each independently a natural or unnatural amino acid;
X and Z are each independently C₁-C₈ alkyl, C₂-C₈ alkenyl, or C₂-C₈ alkynyl, C₁-C₈ heteroalkyl, C₂-C₈ heteroalkenyl, or C₂-C₈ heteroalkynyl, C₁-C₈ alkoxy, or a bond, each of which may be substituted with 0-5 R_{A};
Y is —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl.

In certain embodiments, R″ and R^{y} are H.

In other embodiments, R^{x} is optionally substituted aryl.

In another embodiment, aryl is substituted with optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl, optionally substituted alkyl-heterocyclo; or optionally substituted alkyl-heteroaryl.

In a further embodiment, aryl is substituted with optionally substituted alkyl-heterocyclo or optionally substituted alkyl-heteroaryl.

In still another embodiment, aryl is substituted with

In one embodiment, the invention provides a compound of formula IX:

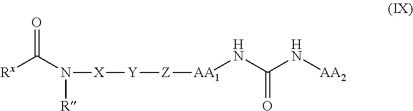

wherein
R″ is H or optionally substituted alkyl;
R^{x} is optionally substituted aryl or optionally substituted alkyl;
AA₁ and AA₂ are each independently a natural or unnatural amino acid;
X and Z are each independently C₁-C₈ alkyl, C₂-C₈ alkenyl, or C₂-C₈ alkynyl, C₁-C₈ heteroalkyl, C₂-C₈ heteroalkenyl, or C₂-C₈ heteroalkynyl, C₁-C₈ alkoxy, or a bond, each of which may be substituted with 0-5 R_{A};
Y is —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl.

In one embodiment, R″ is H.

In another embodiment, R^{x} is optionally substituted alkyl. In a further embodiment, alkyl is substituted with optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl, optionally substituted alkyl-heterocyclo; or optionally substituted alkyl-heteroaryl. In a further embodiment, alkyl is substituted with optionally substituted heterocyclo or optionally substituted heteroaryl.

In certain embodiments, the invention provides for the following compounds:

In another embodiment, the invention provides a compound further comprising a metal.

In another embodiment, the invention provides a compound of formula X:

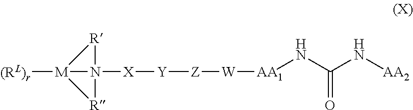

wherein
M is a metal or Al—F;
R^{L} is a metal ligand;
R′ is —CO—NR^{x}R^{y}—, —CS—NR^{x}R^{y}—, COR^{x}, CSR^{x}, C(NR^{x})R^{x}, —S(O)ₚR^{x}—, —CO₂—NR^{x}R^{y}—, or optionally substituted alkyl;
R″ is H or optionally substituted alkyl;
R^{x} is optionally substituted aryl or optionally substituted alkyl;
R^{y} is H, optionally substituted aryl or optionally substituted alkyl;
X and Z are each independently C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, or a bond, each of which may be substituted with 0-5 R_{A};
Y and W are each independently —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl and
r is 1-5.

In certain embodiments, M is AlF, Tc, Re, Ga, Cu, Y, Ac, Bi or In. In a further embodiment, the metal is a radioactive isotope. In still another further embodiment, M is Al F, Tc-99m, Re-188, Re-186, Ga-68, Sc-44, Cu-64, Y-90, Y-86, Ac-225, Bi-213, In-111, Tc-94m, Sm-153, Ho-166, Lu-177, Cu-67, or Dy-166 or paramagnetic metals like Gd or Mn.

In another embodiment, R′ is CO.

In still another embodiment, r is 1-3.

In another embodiment, the invention provides a compound of formula XI:

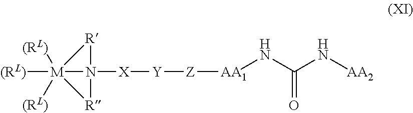

wherein the residues have the same meaning as above with respect to formula (X).

In one aspect, the invention provides a method of imaging in a subject, comprising the steps of:

providing a radiolabeled compound according to Formula X:

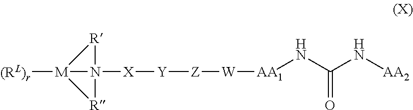

wherein
M is a metal;
R^{L} is a metal ligand;
R′ is —CO—NR^{x}R^{y}—, —CS—NR^{x}R^{y}—, COR^{x}, CSR^{x}, C(NR^{x})R^{x}, —S(O)ₚR^{x}—, —CO₂—NR^{x}R^{y}—, or optionally substituted alkyl;
R″ is H or optionally substituted alkyl;
R^{x} is optionally substituted aryl or optionally substituted alkyl;
R^{y} is H, optionally substituted aryl or optionally substituted alkyl;
X and Z are each independently C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, or a bond, each of which may be substituted with 0-5 R_{A};
Y and W are each independently —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl; and
r is 1-5;
wherein the compound of Formula IX comprises at least one radioisotope; or a pharmaceutically acceptable salt thereof;
contacting cells or tissues with the compound;
detecting the compound in the cells or tissue; and
imaging the compound in the cells or tissue.

In one embodiment, the invention provides a method wherein the metal is Al-F-18, Tc-99m, Re-188, Re-186, Ga-68, Cu-64, Y-90, Y-86, Ac-225, Bi-213, In-111, Tc-94m, Sm-153, Ho-166, Lu-177, Cu-67, or Dy-166 or paramagnetic metals like Gd or Mn.

In another embodiment, the imaging method is suitable for imaging of pain.

In certain embodiments, the radiolabeled compound is stable in vivo.

In other embodiments, the radiolabeled compound is detected by positron emission tomography (PET) or single photon emission computed tomography (SPECT).

In other embodiments, the paramagnetic compound is detected by MR.

In one embodiment, the invention provides a method wherein the subject is a human, rat, mouse, cat, dog, horse, sheep, cow, camel, monkey, avian, or amphibian.

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

The compounds herein described may have one or more charged atoms. For example, the compounds may be zwitterionic, but may be neutral overall. Other embodiments may have one or more charged groups, depending on the pH and other factors. In these embodiments, the compound may be associated with a suitable counter-ion. It is well known in the art how to prepare salts or exchange counter-ions. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Counter-ions may be changed, for example, by ion-exchange techniques such as ion-exchange chromatography. All zwitterions, salts and counter-ions are intended, unless the counter-ion or salt is specifically indicated. In certain embodiments, the salt or counter-ion may be pharmaceutically acceptable, for administration to a subject.

When any variable occurs more than one time in any constituent or formula for a compound, its definition at each occurrence is independent of its definition at every other occurrence. Thus, for example, if a group is shown to be substituted with (X)ₙ, where n is 1, 2, 3, 4, or 5, then said group may optionally be substituted with up to five X groups and each occurrence is selected independently from the definition of X. Also, combinations of substituents and/or variables are permissible only if such combinations result in stable compounds.

As indicated above, various substituents of the various formulae are “substituted” or “may be substituted.” The term “substituted,” as used herein, means that any one or more hydrogens on the designated atom or group is replaced with a substituent, provided that the designated atom's normal valence is not exceeded, and that the substitution results in a stable compound. When a substituent is oxo (keto, i.e., =0), then 2 hydrogens on an atom are replaced. The present invention is intended to include all isotopes (including radioisotopes) of atoms occurring in the present compounds. When the compounds are substituted, they may be so substituted at one or more available positions, typically 1, 2, 3 or 4 positions, by one or more suitable groups such as those disclosed herein. Suitable groups that may be present on a “substituted” group include e.g., halogen; cyano; hydroxyl; nitro; azido; amino; alkanoyl (such as a C₁-C₆ alkanoyl group such as acyl or the like); carboxamido; alkyl groups (including cycloalkyl groups, having 1 to about 8 carbon atoms, for example 1, 2, 3, 4, 5, or 6 carbon atoms); alkenyl and alkynyl groups (including groups having one or more unsaturated linkages and from 2 to about 8, such as 2, 3, 4, 5 or 6, carbon atoms); alkoxy groups having one or more oxygen linkages and from 1 to about 8, for example 1, 2, 3, 4, 5 or 6 carbon atoms; aryloxy such as phenoxy; alkylthio groups including those having one or more thioether linkages and from 1 to about 8 carbon atoms, for example 1, 2, 3, 4, 5 or 6 carbon atoms; alkylsulfinyl groups including those having one or more sulfinyl linkages and from 1 to about 8 carbon atoms, such as 1, 2, 3, 4, 5, or 6 carbon atoms; alkylsulfonyl groups including those having one or more sulfonyl linkages and from 1 to about 8 carbon atoms, such as 1, 2, 3, 4, 5, or 6 carbon atoms; aminoalkyl groups including groups having one or more N atoms and from 1 to about 8, for example I₅ 2, 3, 4, 5 or 6, carbon atoms; carbocyclic aryl having 4, 5, 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, (e.g. benzyl); arylalkoxy having 1 to 3 separate or fused rings and from 6 to about 18 ring carbon atoms (e.g. O-benzyl); or a saturated, unsaturated, or aromatic heterocyclic group having 1 to 3 separate or fused rings with 3 to about 8 members per ring and one or more N, O or S atoms, (e.g. coumarinyl, quinolinyl, isoquinolinyl, quinazolinyl, pyridyl, pyrazinyl, pyrimidyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl). Such heterocyclic groups may be further substituted, e.g. with hydroxy, alkyl, alkoxy, halogen and amino.

As used herein, “alkyl” is intended to include branched, straight-chain, and cyclic saturated aliphatic hydrocarbon groups. Examples of alkyl include, but are not limited to, methyl, ethyl, N-propyl, iso-propyl, n-butyl, sec-butyl, tert-butyl, n-pentyl, and sec-pentyl. In certain embodiments, alkyl groups are C₁-C₆ alkyl groups or C₁-C₄ alkyl groups. Particular alkyl groups are methyl, ethyl, propyl, butyl, and 3-pentyl. The term “C₁-C₆ alkyl” as used herein means straight-chain, branched, or cyclic C₁-C₆ hydrocarbons which are completely saturated and hybrids thereof such as (cycloalkyl)alkyl. Examples of C₁-C₆ alkyl substituents include methyl (Me), ethyl (Et), propyl (including n-propyl (n-Pr, ⁿPr), iso-propyl (i-Pr, ⁱPr), and cyclopropyl (c-Pr, ^{c}Pr)), butyl (including n-butyl (n-Bu, ⁿBu), iso-butyl (i-Bu, ⁱBu), sec-butyl (s-Bu, ^{s}Bu), tert-butyl (t-Bu, ^{t}Bu), or cyclobutyl (c-Bu, ^{c}Bu)), and so forth. “Cycloalkyl” is intended to include saturated ring groups, such as cyclopropyl, cyclobutyl, cyclopentyl, or cyclohexyl. Cycloalkyl groups typically will have 3 to about 8 ring members. In the term “(cycloalkyl)alkyl”, cycloalkyl, and alkyl are as defined above, and the point of attachment is on the alkyl group. This term encompasses, but is not limited to, cyclopropylmethyl, cyclopentylmethyl, and cyclohexylmethyl.

As used herein, “alkenyl” is intended to include hydrocarbon chains of either a straight or branched configuration comprising one or more unsaturated carbon-carbon bonds, which may occur in any stable point along the chain, such as ethenyl and propenyl. Alkenyl groups typically will have 2 to about 8 carbon atoms, more typically 2 to about 6 carbon atoms.

As used herein, “alkynyl” is intended to include hydrocarbon chains of either a straight or branched configuration comprising one or more carbon-carbon triple bonds, which may occur in any stable point along the chain, such as ethynyl and propynyl. Alkynyl groups typically will have 2 to about 8 carbon atoms, more typically 2 to about 6 carbon atoms.

As used herein, “haloalkyl” is intended to include both branched and straight-chain saturated aliphatic hydrocarbon groups having the specified number of carbon atoms, substituted with 1 or more halogen atoms. Examples of haloalkyl include, but are not limited to, mono-, di-, or tri-fluoromethyl, mono-, di-, or tri-chloromethyl, mono-, di-, tri-, tetra-, or penta-fluoroethyl, and mono-, di-, tri-, tetra-, or penta-chloroethyl, etc. Typical haloalkyl groups will have 1 to about 8 carbon atoms, more typically 1 to about 6 carbon atoms.

As used herein, “alkoxy” represents an alkyl group as defined above attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups typically have 1 to about 8 carbon atoms, more typically 1 to about 6 carbon atoms.

As used herein, “haloalkoxy” represents a haloalkyl group as defined above with the indicated number of carbon atoms attached through an oxygen bridge. Haloalkoxy groups will have 1 to about 8 carbon atoms, more typically 1 to about 6 carbon atoms.

As used herein, “alkylthio” includes those groups having one or more thioether linkages and typically from 1 to about 8 carbon atoms, more typically 1 to about 6 carbon atoms.

As used herein, the term “alkylsulfinyl” includes those groups having one or more sulfoxide (SO) linkage groups and typically from 1 to about 8 carbon atoms, more typically 1 to about 6 carbon atoms.

As used herein, the term “alkylsulfonyl” includes those groups having one or more sulfonyl (SO₂) linkage groups and typically from 1 to about 8 carbon atoms, more typically 1 to about 6 carbon atoms.

As used herein, the term “alkylamino” includes those groups having one or more primary, secondary and/or tertiary amine groups and typically from 1 to about 8 carbon atoms, more typically 1 to about 6 carbon atoms.

As used herein, “Halo” or “halogen” refers to fluoro, chloro, bromo, or iodo; and “counter-ion” is used to represent a small, negatively charged species such as chloride, bromide, hydroxide, acetate, sulfate, and the like.

As used herein, “carbocyclic group” is intended to mean any stable 3- to 7-membered monocyclic or bicyclic or 7- to 13-membered bicyclic or tricyclic group, any of which may be saturated, partially unsaturated, or aromatic. In addition to those exemplified elsewhere herein, examples of such carbocycles include, but are not limited to, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, phenyl, naphthyl, indanyl, and tetrahydronaphthyl.

As used herein, the term “aryl” includes groups that contain 1 to 3 separate or fused rings and from 6 to about 18 ring atoms, without hetero atoms as ring members. Example of aryl groups include include but are not limited to phenyl, and naphthyl, including 1-napthyl and 2-naphthyl.

As used herein, “heterocyclic group” is intended to include saturated, partially unsaturated, or unsaturated (aromatic) groups having 1 to 3 (possibly fused) rings with 3 to about 8 members per ring at least one ring containing an atom selected from N, O or S. The nitrogen and sulfur heteroatoms may optionally be oxidized. The term or “heterocycloalkyl” is used to refer to saturated heterocyclic groups.

A heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized.

As used herein, the term “heteroaryl” is intended to include any stable 5- to 7-membered monocyclic or 10- to 14-membered bicyclic heterocyclic aromatic ring system which comprises carbon atoms and from 1 to 4 heteroatoms independently selected from the group consisting of N, O and S. In exemplary embodiments, the total number of S and O atoms in the aromatic heterocycle is not more than 2, and typically not more than 1. Examples of heteroaryl include, but are not limited to, those exemplified elsewhere herein and further include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H.6HA,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Exemplary heteroaryl groups include, but are not limited to, pyridinyl, pyrimidinyl, furanyl, thienyl, pyrrolyl, pyrazolyl, pyrrolidinyl, morpholinyl, piperidinyl, piperazinyl, and imidazolyl.

In certain embodiments, Z is tetrazole or CO₂Q. When Z is tetrazole, the tetrazole ring is attached through the carbon atom.

Certain embodiments include compounds according to formula I where Z is CO₂Q. In other embodiments, Q is hydrogen. In some specific embodiments, Z is CO₂Q and Q is hydrogen.

Certain embodiments include compounds according to formula I, where m is 1, 2, 3, or 4.

Other embodiments include compounds according to formula I wherein m is 0, 1, 2, 3, 4, 5, or 6; R is a pyridine ring selected from the group consisting of

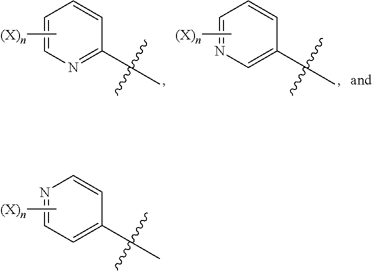

wherein X is a radioisotope of fluorine, a radioisotope of iodine, a radioisotope of bromine, a radioisotope of astatine, —NHNH—CH₂R³. In certain embodiments, n is 1. Each Q is hydrogen; Z is tetrazole or CO₂Q; and R³ is alkyl, alkenyl, alkynyl, aryl, or heteroaryl each of which is substituted by a radioisotope of fluorine, a radioisotope of iodine, a radioisotope of bromine, or a radioisotope of astatine. In certain embodiments, R³ is aryl, substituted by a radioisotope of fluorine, a radioisotope of iodine, a radioisotope of bromine, or a radioisotope of astatine.

Other embodiments include compounds having the structure

wherein m is not 0. R is a pyridine ring selected from the group consisting of

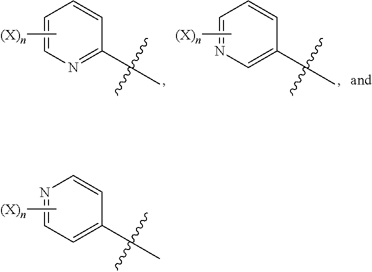

wherein X is a radioisotope of fluorine, a radioisotope of iodine, a radioisotope of bromine, a radioisotope of astatine, or —NHN═CHR³. and R³ is alkyl, alkenyl, alkynyl, aryl, or heteroaryl each of which is substituted by a radioisotope of fluorine, a radioisotope of iodine, a radioisotope of bromine, or a radioisotope of astatine. In certain embodiments, n is 1. Other specific embodiments include compounds where X is a radioisotope of fluorine, a radioisotope of iodine, a radioisotope of bromine, or a radioisotope of astatine. In certain embodiments, R³ is aryl, substituted by a radioisotope of fluorine, a radioisotope of iodine, bromine, a radioisotope of bromine, or a radioisotope of astatine. Specific embodiments include compounds having the structure shown above, where Z is CO₂Q, Q is hydrogen, and m is 4.

Compounds according to this embodiment can be prepared, for example, as disclosed in patent application publications WO 2010/014933 (The Johns Hopkins University),

In some embodiments, the PSMA binding molecule has the general formula (XII):

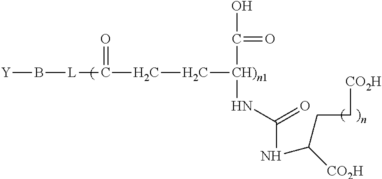

wherein:
n and n¹ are each independently 1, 2, 3, or 4;
L is an optionally substituted aliphatic or heteroaliphatic linking group; B comprises at least one negatively charged amino acid; and Y is a H of B or can include at least one of a detectable moiety, therapeutic agent, or a theranostic agent that is directly or indirectly linked or coupled to B. In other embodiments, Y can be selected from the group consisting of an imaging agent, anticancer agent, or combination thereof.

In other embodiments, L can be an optionally substituted aliphatic or heteroaliphatic group that includes at least one ring selected from the group consisting of an optionally substituted 4 to 7 membered nonaromatic heterocyclic ring and an optionally substituted C4-C7 cycloalkyl ring.

An aliphatic group is a straight chained, branched or cyclic non-aromatic hydrocarbon, which is completely saturated or which contains one or more units of unsaturation. An alkyl group is a saturated aliphatic group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. C1-C4 straight chained or branched alkyl or alkoxy groups or a C3-C8 cyclic alkyl or alkoxy group (preferably C1-C4 straight chained or branched alkyl or alkoxy group) are also referred to as a “lower alkyl” or “lower alkoxy” groups; such groups substituted with —F, —CI, —Br, or —I are “lower haloalkyl” or “lower haloalkoxy” groups; a “lower hydroxyalkyl” is a lower alkyl substituted with —OH; and the like.

Suitable optional substituents for a substitutable atom in alkyl, cycloalkyl, aliphatic, cycloaliphatic, heterocyclic, benzylic, aryl, or heteroaryl groups described herein are those substituents that do not substantially interfere with the activity of the disclosed compounds. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)=), a carbon atom with two valences available (e.g., —C(H₂)—) can form one or two single bonds to one or two substituents (e.g., —C(alkyl)(Br))—, —C(alkyl)(H)—) or a double bond to one substituent (e.g., —C=0)-), and the like. Substitutions contemplated herein include only those substitutions that form stable compounds.

For example, suitable optional substituents for substitutable carbon atoms include —F, —CI, —Br, —I, —CN, —NO₂, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —POR^{a}R^{b}, PO₂R^{a}R^{b}, —PO₃R^{a}R^{b}, —PO₄R^{a}R^{b}, —P(S)R^{a}R^{b}, —P(S)OR^{a}R^{b}, —P(S)O₂R^{a}R^{b}, —P(S)O₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), NR^{c}C(O)R^{a}, —NR^{c}C(O)OR^{a}, —NR^{c}C(O)N(R^{a}R^{b}), —C(NR^{o})—N(R^{a}R^{b}), —NR^{d}—C(NR^{o})—N(R^{a}R^{b}), —NR^{a}N(R^{a}R^{b}), —CRC═CR^{a}R^{b}, —C═CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, wherein R^{a}—R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group. Also contemplated are isomers of these groups.

Suitable substituents for nitrogen atoms having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), SO₂N(R^{a}R^{b}), —NR^{c}C(O)R^{a}, —NR^{c}C(O)OR^{a}, —NR^{c}C(O)N(R^{a}R^{b}), and the like.

Suitable substituents for nitrogen atoms having three covalent bonds to other atoms include —OH, alkyl, and alkoxy (preferably C1-C4 alkyl and alkoxy). Substituted ring nitrogen atoms that have three covalent bonds to other ring atoms are positively charged, which is balanced by counteranions such as chloride, bromide, fluoride, iodide, formate, acetate and the like. Examples of other suitable counter anions are provided in the section below directed to suitable pharmacologically acceptable salts.

other embodiments, B can include at least one, two, three, four, or more negatively charged amino acids, i.e., amino acids with a negative charged side chain, such as glutamic acid, aspartic acid, and/or tyrosine. B can also include other amino acids that facilitate binding of B to Y and/or the PSMA ligand (or PSMA-binding molecule) to a detectable moiety, therapeutic agent, and/or theranostic agent.

In some embodiments, B can have the following formula:

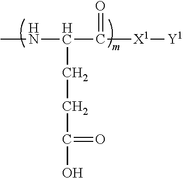

wherein m is 1, 2, 3, or 4, X¹ is an amino acid, and Y¹ is a H of X¹ or includes at least one of an amino acid, peptide, detectable moiety, therapeutic agent, or theranostic agent that is directly or indirectly linked to X¹.

In certain embodiments, X¹ can facilitate binding of B to Y and/or the PSMA-binding molecule to a detectable moiety, therapeutic agent, and/or theranostic agent.

In other embodiments, B can have the following formula:

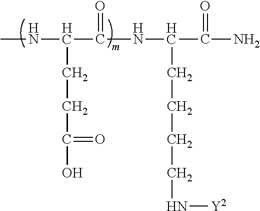

wherein m is 1, 2, 3, or 4 and Y² is a H or can include at least one of an amino acid, peptide, detectable moiety, therapeutic agent, or theranostic agent.

In other embodiments, the compound can have the general formula:

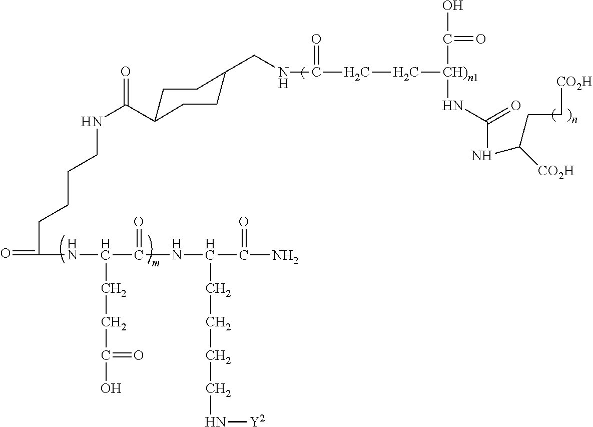

wherein m, n, and n¹ are independently 1, 2, 3, or 4; and Y² is a H or can include at least one of an amino acid, peptide, detectable moiety, therapeutic agent, or theranostic agent.

In some embodiments, Y, Y¹, or Y² can be a detectable moiety that is directly or indirectly coupled to B or the PSMA ligand (i.e. the PSMA-binding molecule). Examples of detectable moieties include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, chelating groups, and biotin, dioxigenin or other haptens and proteins for which antisera or monoclonal antibodies are available.

Other suitable PSMA-binding molecules are disclosed in publications WO2012174136 (paragraph [0013]) and WO2015055318 (formulae Ia and Ib) and are hereby explicitly incorporated by reference.

Other embodiments of the inventions include methods of imaging one or more cells, organs or tissues comprising exposing cells to or administering to a subject, e.g. a patient suffering from pain, an effective amount of a PMSA-binding agent with an isotopic label suitable for imaging.

The imaging methods of the invention are suitable for imaging physiological process associated with the development or maintenance of pain in which PSMA is involved. Typically, imaging methods are suitable for identification of areas of tissues or targets, particularly in a patient suffering from pain, which express high concentrations of PSMA.

In certain embodiments, the radiolabeled compound is detected by positron emission tomography (PET) or single photon emission computed tomography (SPECT).

In one embodiment, the invention provides a method wherein the subject is a mammal, e.g. a human, or a companion or domestic animal.

Other embodiments provide kits comprising a compound according to the invention. In certain embodiments, the kit provides packaged pharmaceutical compositions comprising a pharmaceutically acceptable carrier and a compound of the invention. In certain embodiments the packaged pharmaceutical composition will comprise the reaction precursors necessary to generate the compound of the invention upon combination with a radiolabeled precursor.

Other packaged pharmaceutical compositions provided by the present invention further comprise indicia comprising at least one of: instructions for preparing compounds according to the invention from supplied precursors, instructions for using the composition to image cells or tissues expressing PSMA in a patient suffering from a pain, or instructions for using the composition to image pain.

In certain embodiments, a kit according to the invention contains from about 1 to about 30 mCi of the radionuclide-labeled imaging agent described above, in combination with a pharmaceutically acceptable carrier. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like. The kit may provide a compound of the invention in solution or in lyophilized form, and these components of the kit of the invention may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form. Determination and optimization of such stabilizers and stabilization methods are well within the level of skill in the art. In certain embodiments, a kit provides a non-radiolabeled precursor to be combined with a radiolabeled reagent on-site. Examples of radioactive reagents include Al[¹⁸F], Na[¹²⁵I], Na[¹³¹I], Na[¹²³I], Na[¹²⁴I], K[¹⁸F], Na[⁷⁶Br], Na[⁷⁵Br], Na[²¹¹At]. Other radiolabeled reagents include activated radiolabeled benzoyl compounds, radiolabeled pyridine carboxylates, radiolabeled bromomethyl pyridine compounds, and radiolabeled aldehydes discussed previously.

Imaging agents of the invention may be used in accordance with the methods of the invention by one of skill in the art. Images can be generated by virtue of differences in the spatial distribution of the imaging agents which accumulate at a site when contacted with PSMA. The spatial distribution may be measured using any means suitable for the particular label, for example, a gamma camera, a PET apparatus, a SPECT apparatus, and the like. The extent of accumulation of the imaging agent may be quantified using known methods for quantifying radioactive emissions.

In general, a detectably effective amount of the imaging agent of the invention is administered to a subject. In accordance with the invention, “a detectably effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, and the dosimetry. Detectably effective amounts of the imaging agent of the invention can also vary according to instrument and film-related factors. Optimization of such factors is well within the level of skill in the art. The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

A “pharmaceutically acceptable carrier” refers to a biocompatible solution, having due regard to sterility, p[Eta], isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial and antifungal agents, isotonic agents, and the like. The pharmaceutically acceptable carrier may also contain stabilizers, preservatives, antioxidants, or other additives, which are well known to one of skill in the art, or other vehicle as known in the art.

As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Lists of additional suitable salts may be found, e.g., in Remington's Pharmaceutical Sciences, 17th ed., Mack Publishing Company, Easton, Pa., p. 1418 (1985).

The herein described compounds may be used in methods of diagnosing pain, preferably in a patient suffering from diseases or disorders that can be associated with pain. The herein compounds may also be used in methods of imaging pain, preferably in a patient suffering from diseases or disorders that can be associated with pain, and more preferably in patients suffering from pain or those that are suspected to suffer from pain, but are not able to communicate, e.g. patients with dementia, children, unconscious patients, etc. It is possible to use the methods more than once in order to monitor the development of pain. The herein compounds may also be used in methods of imaging the site of pain and/or the source of pain, wherein the PSMA-binding agents of the invention localize specifically to said site of pain. Preferably, a control of the methods is used, e.g. when the left is painful and the right one is not, it may be preferred to compare the respective sites in order to decide whether or not a PMSA-binding molecule as defined above localizes specifically to the affected site. The staining intensities of affected versus non-affected areas of the body provide can be compared in attempts to decide whether or not a localization of the PSMA-binding molecule as defined herein is specific or not. The intensity of signals as measured with the imaging methods used according to the invention provides guidance on the specificity of the binding of the herein disclosed compounds. Preferably, the intensity of signals derived from the detectable unit of the PSMA-binding molecule can be allocated to statistically reliable information obtained using respective statistic methods.

The contents of all cited references (including literature references, issued patents, published patent applications) as cited throughout this application are hereby expressly incorporated by reference. The invention and the manner and process of making and using it, are described in such full, clear, concise and exact terms as to enable any person skilled in the art to which it pertains, to make and use the same.

It is to be understood that the foregoing describes exemplary embodiments of the present invention and that modifications may be made therein without departing from the spirit or scope of the present invention as set forth in the appended claims.

### EXAMPLES

### 1. Material and Methods

### 1.1. Animals

Experiments were performed in male 6- to 12-week-old C57BL/6 mice. Animals were housed on a 12/12 light/dark cycle with access to food and water ad libitum. All experiments adhered to the guidelines of the International Association for the Study of Pain and to the ARRIVE guidelines, and were approved by our local Ethics Committee for Animal Research.

### 1.2. Complete-Freund's-Adjuvant Model of Inflammatory Pain

The mechanical sensitivity of the plantar side of a hindpaw was assessed with an automated testing device (dynamic plantar aesthesiometer; Ugo Basile). This device pushes a thin probe (0.5 mm diameter) with increasing force through a wire-grated floor against the plantar surface of the paw from beneath, and it automatically stops and records the latency time after which the animal withdraws the paw. The force increased from 0 to 5 g within 10 s (0.5 g/s ramp) and was then held at 5 g for an additional 10 s (Schmidtko et al., 2008a). The paw withdrawal latency was taken to be the mean of three consecutive trials with at least 10 s in between. After baseline measurements, 20 al of complete Freund's adjuvant (CFA; containing 1 mg/ml heat-killed Mycobacterium tuberculosis in paraffin oil 85% and mannide monooleate 15%; Sigma-Aldrich) was injected into the plantar subcutaneous space of a hindpaw, and paw withdrawal latencies were determined at the indicated timepoints after CFA injection (Schmidtko et al., 2008b).

### 1.3. Sciatic Nerve Lesion Model of Neuropathic Pain

Under isoflurane anesthesia, the tibial and common peroneal branches of the sciatic nerve were ligated and sectioned distally, while the sural nerve was left intact (Decosterd and Woolf, 2000); (Bourquin et al., 2006). Mechanical hypersensitivity at the lateral surface of the hindpaw (sural nerve skin area) was determined using a Dynamic Plantar Aesthesiometer as described above.

### 1.4. ¹⁸F-DCFPyL Production

^{[18F]}Fluoride was produced via the 180 (p,n)18F reaction by bombardment of enriched [18O] water with 16.5 MeV protons using a MC16 cyclotron (Scanditronix, Uppsala, Sweden) at the Max Planck Institute for Metabolism Research. The synthesis of [¹⁸F]DCFPyL was performed under GMP conditions as previously reported by Chen et al. (Chen et al., 2011). The radiolabeled product was analyzed using the following conditions: column: Chromolith SpeedROD®, 50×4.6 mm (Merck Millipore, Darmstadt, Germany); eluent: 5% EtOH in 0.38% H₃PO₄ (pH 2); flow rate: 3.0 ml/min; tR=2.2 min. The final product was formulated in a PBS solution (pH 4-6). The formulated solution of [¹⁸F]DCFPyL was tested for sterility and endotoxin content. Production under GMP conditions provided [¹⁸F]DCFPyL in reasonable radiochemical yields of 8-12% and in high radiochemical purity (99%). The specific activity of [¹⁸F]DCFPyL amounted to 72 GBq/μmol. The PSMA enzyme inhibition potency of [¹⁸F]DCFPyL was determined with a modified Amplex Red glutamic acid assay after incubation with the cell lysates of LNCaP cell extracts in the presence of NAAG for 2 h at 37° C. The enzyme inhibitory constant (Ki) for [¹⁸F]DCFPyL was 1.1±0.1 nmol/l, comparable with that of ZJ-43, which was 1.4±0.2 nmol/l under the same measurement conditions. ZJ-43 is a urea-based potent inhibitor of NAAG and is used as an internal reference in the assay.

### 1.5. Micro-PET Analysis

Mice were anesthetized in pairs (initial dosage: 5% isoflurane in O₂/air (3:7), reduced to 1.5-2.5% for maintenance), and 10 MBq [¹⁸F]DCFPyL in a volume of 250 μl of 10% ethanolic isotonic saline was injected into the lateral tail vein of each mouse. The animals were allowed to wake up in their home cage, where they remained for 50 min. Subsequently, mice were reanesthetized, killed to reduce the time of procedures on the living animal, and placed on a two-animal holder (Medres®). PET scans in list mode were performed using a Focus 220 micro PET scanner (CTI-Siemens®) with a resolution at center of field of view of 1.4 mm. Data acquisition started exactly one hour after [¹⁸F]DCFPyL-injection and lasted 60 min. It was followed by a transmission scan using a ⁵⁷Co-point source for attenuation correction. Following Fourier rebinning, data were reconstructed using the iterative OSEM3D/MAP procedure (Qi et al., 1998) resulting in voxel sizes of 0.38×0.38×0.80 mm. Images were Gauss-filtered (1.5 mm FWHM) and displayed as % injected dose (% ID).

Four individual volumes of interest (VOIs, 24 mm³ each) were drawn, two of which were placed over the left and right sciatic nerve plexus. The other two were positioned either over the sciatic nerve lesion in the thigh and the undamaged contra-lesional thigh (in case of SNI model), or over the hindpaws (in case of CFA model). We therefore received a mean value of [¹⁸F]DCFPyL binding (unit: % ID) for each affected area and its contralateral unaffected counterpart per animal. The ratio of ipsi- and contralateral VOIs was calculated.

### 1.6. Statistics

Animals were grouped according to the number of days after SNI- or CFA-intervention, and the [¹⁸F]DCFPyL binding ratio was compared using a one-way ANOVA. Furthermore, association of [¹⁸F]DCFPyL binding ratio with individual pain ratio (ipsi-/contralateral) was determined using a Pearson correlation test.

### 2. Results

PSMA is a classical target for the diagnosis of various cancers, which strongly overexpress this enzyme. If PSMA can also be used for the detection of pain originating lesions at potentially much smaller and potentially much lower PSMA concentrations has so far not been investigated. To establish proof of principle, we tested the PSMA PET-tracer [¹⁸F]DCFPyL (enrichment of the detectable unit of the PSMA-binding molecule) in two classical models of pain: 1) Sciatic nerve injury as a model of neuropathic pain, and 2) CFA-induced inflammatory pain.

### 2.1. PSMA-Binding Entities Visualize the Location of CFA-Induced Inflammatory Pain

The CFS-induced inflammatory pain model has been performed as described in the Material and Method section. As a consequence of the injection, the threshold for mechanical stimuli drops drastically resulting in mechanical hyperalgesia. To assure the onset of hyperalgesia, animals were tested with the dynamic plantar aesthesiometer. Each animal was tested at the treated hind paw as well as the contralateral sham-treated paw.

As commonly reported for CFA-mediated mechanical hyperalgesia, strong hyperalgesia 2 days after the injection of CFA into the hindpaw was observed. After that hyperalgesia decreased over the next 2 weeks as measured at day 7 and day 14 (see FIG. 1).

Each measurement was taken on a separate set of animals. After measurements, the animals were injected with 10 MBq of [¹⁸F]DCFPyL into the tail vene. To simplify the measurement methodology, animals were sacrificed instead of anaesthesized after 1 h and measured in a iPET. Images were analyzed for the section of maximal intensity at the site of injection, the respective areas quantified, and the ratio between ipsi- and contralateral side calculated. A high [¹⁸F]DCFPyL uptake was observed at the site of injection while there was only little to no uptake at the contralateral side (white arrows in FIG. 2).

The quantification of the uptake showed in average a 2-fold increase over controls. There was a marked variability between the individual animals. (see FIG. 3).

2.2. PSMA-Selective Ligands (i.e. PSMA-Binding Molecules with Detectable Unit) Allow to Visualize the Location of SNI-Induced Neuropathic Pain

The sciatic nerve injury induced neuropathic pain model has been performed as described in the Material and Method section. As a consequence of the injury, the threshold for mechanical stimuli dropped drastically resulting in mechanical hyperalgesia. To assure the onset of hyperalgesia, animals were tested with the dynamic plantar aesthesiometer. Each animal was tested at the treated hind paw as well as the contralateral sham-treated paw.

As commonly reported for SNI-induced mechanical hyperalgesia, we found increasingly strong hyperalgesia over the course of 2 weeks after the injury to the sciatic nerve. It is well established that thereafter the mechanical hyperalgesia remains constant, thus measurements were taken only until two weeks after injury (see FIG. 4).

For each measurement a separate set of animals was used. The animals were injected with 10 MBq of [¹⁸F]DCFPyL into the tail vene. To simplify the measurement methodology, animals were sacrificed instead of anaesthesized after 1 h and measured in a pPET. Images were analyzed for the section of maximal intensity at the site of injection, the respective areas quantified, and the ratio between ipsi- and contralateral side was calculated. We observed a high [¹⁸F]DCFPyL uptake at the site of injury while there was little to no uptake at the contralateral side and little in sham treated animals (compare area at white arrow of FIG. 5 sham vs. SNI).

The quantification of the uptake shows in average a 2-3-fold increase over sham-operated controls (see FIG. 6).

2.3. PSMA-Selective Ligands (i.e. PSMA-Binding Molecules with Detectable Unit) Allow to Visualize the Sensitivity to Pain

The PSMA-selective ligand showed clear enrichment at the site of lesion (CFA and SNI). On average, tracer enrichment was rapid resulting in a maximal intensity plateau already at the earliest time point measured. Nevertheless, the tracer enrichment varied from animal to animal (see FIG. 3 and FIG. 6) as did the individual hyperalgesia (see FIG. 1 and FIG. 4). Therefore, we next tested if there is a correlation of PSMA-ligand enrichment and the respective degree of sensitization. Indeed, correlating the individual measurements (but not the averaged data), there was a strong correlation between radiotracer-enrichment and pain sensitivity. This was true for the CFA-induced mechanical hyperalgesia (see FIG. 7) as well as for the SNI-induced mechanical hyperalgesia (see FIG. 8).

2.4. PSMA-Selective Ligands (i.e. PSMA-Binding Molecules with Detectable Unit) Allow to Visualize the Aetiology of Pain

Our data presented above show enrichment of PSMA-ligads at the site of lesion of inflammatory pain as well as neuropathic pain. Enrichment also correlated with the degree of sensitivity suggesting the usability of the tracer-enrichment as objectifiable measurement of pain. A third aspect of crucial importance in pain is the differentiation between varying aetiologies of pain. Our current data show, that pain originating at a peripheral site can be visualized by PSMA-ligand enrichment. Some pain originates not from the periphery but from changes in the brain. As there are no peripheral lesion sites under these circumstances, a differentiation between these two forms is the necessary consequence of our results.

Peripheral pain can be further differentiated in inflammatory pain versus neuropathic pain. To test, if the enrichment of the PSMA-binding molecule allows differentiation between these two aetiologies, we analyzed the signal at the site of injury as well as along the nerve plexus connecting the site of injury with the spinal cord. As shown above, there is enrichment at the site of lesion but not along the neuronal plexus in animals with inflammatory pain (see FIG. 2). In contrast in animals with neuropathic pain there is also tracer enrichment (enrichment of the detectable unit of the PSMA-binding molecule) along the nerve plexus of the injured nerve was detected (see FIG. 5 and FIG. 6). Additionally, the correlation of enrichment and pain sensitivity in the nerve plexus was even more accurate than for the site of lesion (see FIG. 8) while it was absent in inflammatory pain.

Accordingly, the tracer enrichment (enrichment of the detectable unit of the PSMA-binding molecule) at the nervus plexus appears to be an indicator which enables to differentiate between neuropathic and inflammatory pain.

2.5. PSMA-Selective Ligands (i.e. PSMA-Binding Molecules with Detectable Unit) Allow the Identification of Locations of Pain in Humans.

PSMA-ligands are used for the detection of prostate cancer and its metastasizes in humans. The PSMA-tracer intensity of each dorsal root ganglion of patients with no overt metastasizes was analyzed. Upon normalization to e.g. the uptake signal of the gluteus and averaging the intensities of five patients all dorsal root ganglia uptake values appeared as fairly constant.

Further, a patient diagnosed by the pain center at the university hospital of Cologne to suffer from chronic lower back pain at the lumbo-sacral transition zone with a numeric rating scale value of 7 (out of 10 with 10 being the most excruciating pain possible) increasing to 9 if the patient had to carry anything was subjected to an analysis. The patient was a former steal worker who has been unable to carry even light weights for 4 years. Upon quantification of the normalized dorsal root ganglia tracer intensities significantly increased tracer enrichment in comparison to the control patients were found. Corresponding with the reported pain location, increased values were found particularly in lumbal segments L3-L5. These values were more than two-times the standard deviation higher than the average control patients' values.

Further, a strong enrichment of the PSMA Tracer in thoracic and cervical dorsal root ganglia was detected. The increase was particularly pronounced in the dorsal root ganglion of the first thoracic segment, which is known to innervate the right inner arm. Interestingly, the classical pain anamnesis only identified the lower back pain. However, upon questioning the patient acknowledged chronic pain also in neck and shoulders with an especially pronounced pain in the right inner arm. These findings represent surprising evidence that PSMA-selective ligands identify nerves involved in chronic pain in an patient independent manner.

2.6. PSMA-Selective Ligands (i.e. PSMA-Binding Molecules with Detectable Unit) Allow the Identification of Pain with Diffuse Whole Body Pain.

A number of pain conditions exist, where the pain has not been caused by a local injury or a local change but rather appears as a diffuse whole body pain. It was tested if PSMA-selective ligands can identify such conditions. Comparing the quantified and normalized dorsal root ganglia uptake values of a fibromyalgia patient to the respective values of control patients, a clear increase of intensities above the control patients' values for a large number of body segments corresponding with the diffuse whole body nature of fibromyalgia pain was found (FIG. 10). This provides further surprising evidence for the fact that not only localized, but also diffuse pain can be identified by PSMA-selective ligands.

2.7. PSMA-Selective Ligands (PSMA-Binding Molecules with Detectable Unit) Allow the Identification of Locations of Pain in Animals.

Both pain sensitivity and PSMA-tracer uptake were studied in two different mouse models for the analysis of pain.

In the inflammatory pain model, “Complete Freunds Adjuvant (CFA)” was injected in the left hind paw, which leads to long-lasting inflammation and pain. To induce neuropathic pain in the “Spared nerve injury (SNI) model”, two branches of the sciatic nerve were ligated, while the third branch (the sural nerve) was left intact. In both models, pain sensitivity was measured using the Dynamic Plantar Test at the day before the PET scan. A movable force actuator was positioned below the plantar surface of the animal. A thin (0.5 mm) filament exerted increasing force, until the animal twitched its paw. The time it takes for the animal to retract its paw inversely reflects touch sensitivity.

On the next day, the PSMA tracer [¹⁸F]DCFPyL was intravenously injected. After an uptake period of 60 min, an emission scan was performed for 30 min. Tracer uptake at the lesion site (measured as ratio between ipsi- and contralateral side) was significantly correlated to pain sensitivity (also measured as ipsi-/contralateral ratio). The results are shown in FIGS. 11 and 12.

### Embodiments of the Invention

1. A PSMA-binding molecule comprising a detectable unit for use in the diagnosis and/or imaging of pain in a patient suffering from pain or in a patient that is suspected to suffer from pain.
2. The PSMA-binding molecule comprising a detectable unit for use in the diagnosis and/or imaging of pain, wherein said patient suspected to suffer from pain is unable to communicate verbally.
3. The PSMA-binding molecule according to embodiment 1 or 2, wherein the detectable unit has a structure depicted in formula Compound I

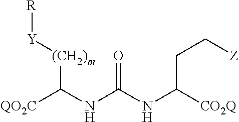

wherein
Z is tetrazole or CO₂Q;
each Q is hydrogen; and

wherein
(A) m is 0, 1, 2, 3, 4, 5, or 6;
R is a pyridine ring selected from the group consisting of

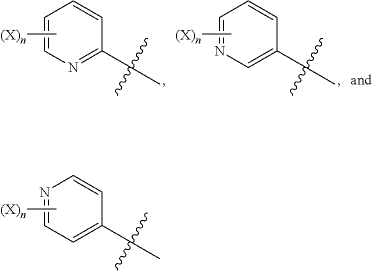

wherein X is a radioisotope of fluorine, a radioisotope of iodine, a radioisotope of bromine, a radioisotope of astatine, —NHN═CHR³, CH₂R³;
n is 1, 2, 3, 4, or 5;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R³ is alkyl, alkenyl, alkynyl, aryl, or heteroaryl each of which is substituted by a radioisotope of fluorine, a radioisotope of iodine, a radioisotope of bromine, or a radioisotope of astatine.
4. The PSMA-binding molecule for use in diagnosis and/or imaging of pain in a subject suffering from pain or in a patient that is suspected to suffer from pain according to embodiments 1 to 3, wherein Z is CO₂Q.
5. The PSMA-binding molecule for use in diagnosis and/or imaging of pain in a subject suffering from pain or in a patient that is suspected to suffer from pain according to embodiment 1 to 4, wherein Q is hydrogen.
6. The PSMA-binding molecule for use in diagnosis and/or imaging of pain in a subject suffering from pain or in a patient that is suspected to suffer from pain according to any one of embodiments 1-5, where m is 1, 2, 3, or 4.
7. The PSMA-binding molecule for use in diagnosis and/or imaging of pain in a subject suffering from pain or in a patient that is suspected to suffer from pain according to any one of embodiments 1-5, having the structure

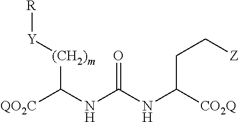

wherein

m is 0, 1, 2, 3, 4, 5, or 6;

R is a pyridine ring selected from the group consisting of

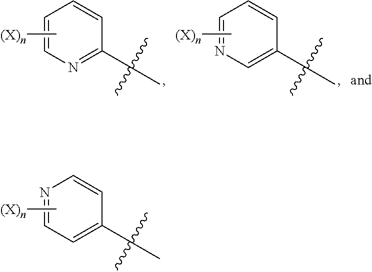

wherein X is a radioisotope of fluorine, a radioisotope of iodine, a radioisotope of bromine, a radioisotope of astatine, —NHN═CHR³;

each Q is independently selected from hydrogen or a protecting group;
Y is O, S, N(R′), C(O), NR′C(O), C(O)N(R′), OC(O), C(O)O, NR′C(O)NR′, NR′C(S)NR′, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R is hydrogen, alkyl, aryl or heteroaryl, each of which may be substituted;
Z is tetrazole or CO₂Q;
R² is C₁-C₆ alkyl; and
R³ is alkyl, alkenyl, alkynyl, aryl, or heteroaryl, each of which is substituted by fluorine, iodine, a radioisotope of fluorine, a radioisotope of iodine, chlorine, bromine, a radioisotope of bromine, or a radioisotope of astatine; NO₂, NH₂, N⁺(R²)₃, Sn(R²)₃, Si(R²)₃, Hg(R²), or B(OH)₂.
8. The PSMA-binding molecule for use in diagnosis and/or imaging of pain in a subject suffering from pain or in a patient that is suspected to suffer from pain according to embodiment 7, having the structure

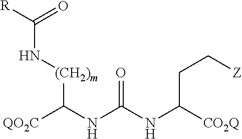

wherein m is not 0.
9. The PSMA-binding molecule for use in diagnosis and/or imaging of pain in a subject suffering from pain or in a patient that is suspected to suffer from pain according to embodiments 7 and 8, where Z is CO₂Q, Q is hydrogen, and m is 4.
10. The PSMA-binding molecule for use in diagnosis and/or imaging of pain in a subject suffering from pain or in a patient that is suspected to suffer from pain according to embodiment 7, having the structure

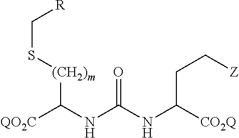

wherein m is not 0.
11. The PSMA-binding molecule according to embodiment 1 for use in diagnosis and/or imaging of pain in a subject suffering from pain or in a patient that is suspected to suffer from pain according to embodiment 10, where Z is CO₂Q, Q is hydrogen, and m is 1, 2, or 3.
12. The PSMA-binding molecule according to embodiment 1 for use in diagnosis and/or imaging of pain in a subject suffering from pain or in a patient that is suspected to suffer from pain according to any one of embodiments 1-6, wherein m is 0, 1, 2, 3, 4, 5, or 6;
   Y is O, S, N(R′), C(O), NR1C(O), C(O)N(R′), OC(O), C(O)O, NR′C(O)NR′, NR′C(S)NR, NR′S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
   R is

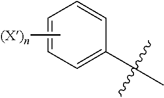

wherein
X1 is selected from the group consisting of NHNH₂, —NHN═CHR³, —NHNH—CH₂R³; wherein R³ is alkyl, alkenyl, alkynyl, aryl, or heteroaryl, each of which is substituted by fluorine, iodine, a radioisotope of fluorine, a radioisotope of iodine, bromine, a radioisotope of bromine, or a radioisotope of astatine; NO₂, NH₂, N⁺(R²)₃, Sn(R²)₃, Si(R²)₃, Hg(R²), and B(OH)₂, where R² is C₁-C₆ alkyl; n is 1, 2, 3, 4, or 5.
13. The PSMA-binding molecule for use in diagnosis and/or imaging of pain in a subject suffering from pain or in a patient that is suspected to suffer from pain according to any one of embodiments 1-12, wherein n is 1.
14. The PSMA-binding molecule for use in diagnosis and/or imaging of pain in a subject suffering from pain or in a patient that is suspected to suffer from pain according to any one of embodiments 1-13, wherein X or X′ is fluorine, iodine, or a radioisotope of fluorine or iodine, bromine, a radioisotope of bromine, or a radioisotope of astatine.
15. The PSMA-binding molecule for use in diagnosis and/or imaging of pain in a subject suffering from pain or in a patient that is suspected to suffer from pain according to any one of embodiments 1-13, wherein X or X′ is fluorine, iodine, or a radioisotope of fluorine or iodine.
16. The PSMA-binding molecule for use in diagnosis and/or imaging of pain in a subject suffering from pain or in a patient that is suspected to suffer from pain according to any one of embodiments 1-6, wherein m is 4, Y is NR′, and R is

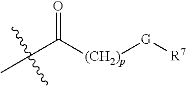

wherein G is O, NR′ or a covalent bond
p is 1, 2, 3, or 4, and
R⁷ is selected from the group consisting of NH₂, N═CHR³, NH—CH₂R³, wherein R³ is alkyl, alkenyl, alkynyl, aryl, heteroaryl each of which is substituted by fluorine, iodine, a radioisotope of fluorine, a radioisotope of iodine, chlorine bromine, a radioisotope of bromine, or a radioisotope of astatine NO₂, NH₂, N⁺(R₂)³, Sn(R²)₃, Si(R²)₃, Hg(R²), and B(OH)₂, wherein R² is C₁-C₆ alkyl.
17. The PSMA-binding molecule for use in diagnosis and/or imaging of pain in a subject suffering from pain or in a patient that is suspected to suffer from pain according to embodiment 16, wherein G is O or NR′.
18. The PSMA-binding molecule for use in diagnosis and/or imaging of pain in a subject suffering from pain or in a patient that is suspected to suffer from pain according to any one of embodiments 1-17, wherein R comprises a radioisotope.
19. The PSMA-binding molecule for use in diagnosis and/or imaging of pain in a subject suffering from pain or in a patient that is suspected to suffer from pain according to embodiment 18, wherein the radioisotope is selected from the group consisting of ¹⁸F, ⁶⁸Ga, ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br and ²¹¹At.
20. The PSMA-binding molecule for use in diagnosis and/or imaging of pain in a subject suffering from pain or in a patient that is suspected to suffer from pain according to embodiments 1 to 3 selected from the group consisting of

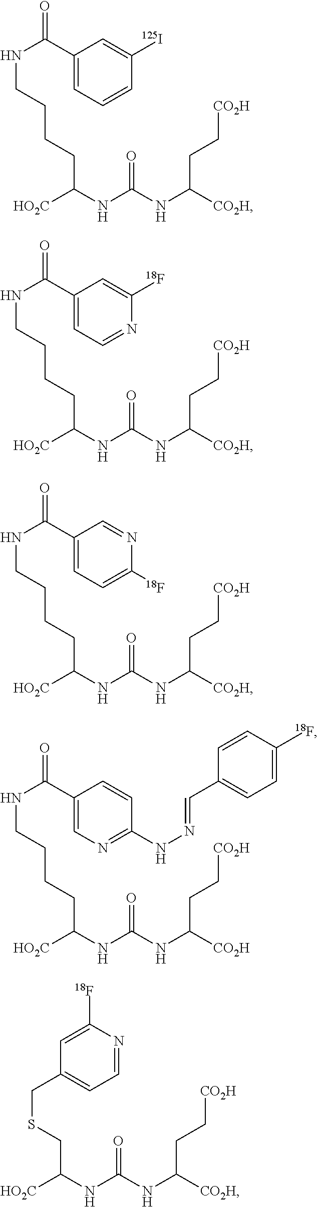

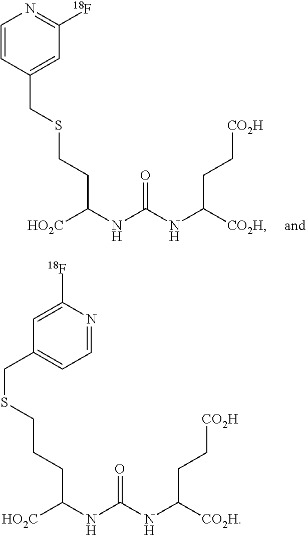

21. The PSMA-binding molecule for use in diagnosis and/or imaging of pain in a subject suffering from pain or in a patient that is suspected to suffer from pain according to embodiments 1 to 3, having the structure

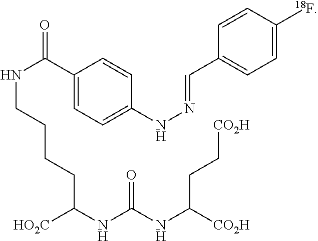

22. The PSMA-binding molecule for use in diagnosis and/or imaging of pain in a subject suffering from pain or in a patient that is suspected to suffer from pain according to embodiments 1 to 3, having the structure

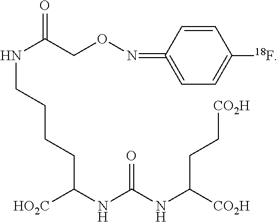

23. The PSMA-binding molecule as defined in any of the preceding embodiments for use in diagnosis or imaging of pain, wherein the pain eliciting location is visualized.
24. The PSMA-binding molecule as defined in any of the preceding embodiments for use in diagnosis or imaging of pain in a subject suffering from pain or in a patient that is suspected to suffer from pain according to any of the preceding embodiments, wherein the level of enzyme PSMA is increased at a site of pain along a peripheral nerve or parts thereof.
25. The PSMA-binding molecule for use in diagnosis or imaging of pain in a subject suffering from pain or in a patient that is suspected to suffer from pain according to embodiment 22, wherein the increased level of enzyme PSMA at said site of pain is detected as intensity of said tracer compound I after administration to said subject and wherein said tracer compound intensity at the site of pain is statistically increased in comparison to a) said tracer compound intensity at the site of an unaffected contralateral site and/or b) to a threshold that has been statistically determined.
26. The PSMA-binding molecule for use in diagnosis or imaging of pain in in a subject suffering from pain or in a patient that is suspected to suffer from pain according to any of the preceding embodiments, wherein diagnosis or imaging of pain may be the visualization of the pain eliciting location, the determination of pain sensitivity, and/or the determination of the aetiology of pain.
27. The PSMA-binding molecule for use in diagnosis or imaging of pain in a subject suffering from pain according or in a patient that is suspected to suffer from pain to any of the preceding embodiments, wherein it is differentiated between peripherally caused pain (peripheral pain) versus central and periphery independent pain.
28. The PSMA-binding molecule for use in diagnosis or imaging of pain in a subject suffering from pain or in a patient that is suspected to suffer from pain according to any of the preceding embodiments, wherein it is determined whether said subject suffers from inflammatory pain or neuropathic pain.
29. The PSMA-binding molecule according to any one of embodiments 1 to 23 in the manufacture of a kit for the diagnosis and/or imaging of pain in a patient suffering from pain according or in a patient that is suspected to suffer from pain to any of the preceding embodiments.
30. A kit comprising a container comprising PSMA-binding molecule as defined in any one of the preceding embodiments, for the diagnosis and/or imaging of pain, optionally comprising instructions for use, and further optionally comprising information on the interpretation of imaging results.
31. A method for diagnosing or imaging of pain in a subject suffering from pain or in a patient that is suspected to suffer from pain comprising administering to said subject an effective amount of a compound according to any of embodiments 1-23.
32. An in vitro method of imaging cells, organs, tissue samples, wherein the cells, organs or tissue samples are exposed to a chemical or physical stimulus suspect to be involved in the development or reduction of pain, and the expression and/or quantity of PSMA is determined using a PSMA-binding molecule as defined in embodiments 1 to 23.

### FIGURE DESCRIPTION

FIG. 1: Injection of CFA into the left hindpaw of adult rat results in the onset of mechanical hyperalgesia. This hyperalgesia is maximal at day 2 and decreases then over the following weeks as reported by others. Data presented are the ratios of the threshold measurements recoded with the dynamic plantar aesthesiometer of the ipsi versus contralateral side (n=2 per timepoint)

FIG. 2: Representative image of the hindlegs of CFA injected mice. CFA was injected into the left paw resulting in pronounced hyperalgesia. Accordingly, we detect strong increase of tracer enrichment at the site of injection in the left paw (left arrow) but not in the right paw (right arrow). Enrichment along the nerve was not apparent.

FIG. 3: Quantification of the enrichment at the side of CFA injection showed nearly 2-fold increase over the contra-lateral side.

FIG. 4: Sciatic nerve injury at the left hindpaw of adult rat results in the onset of mechanical hyperalgesia. This hyperalgesia is maximal at day 14 and then stays constant for the following days. Data presented are the ratios of the threshold measurements recoded with the dynamic plantar aesthesiometer of the ipsi versus contralateral side (n=3 per timepoint)

FIG. 5: [¹⁸F]DCFPyL uptake was measured and visualized. Here represented are imaging sections through the site of injury in sham and operated animals. The white arrow indicates the site of the sham operation or the sectioned sciatic nerve, respectively. The location of sciatic nerve lesion shows strong enrichment in tracer (red area at arrow). But it also shows enrichment along the nerve toward the spinal cord, the so called plexus. Strong enrichment was also detected at the site of tracer injection at the tail as well as along the spinal cord at the center of the image.

FIG. 6: The enrichment of tracer at the site of lesion (left) and along the plexus (right).

FIG. 7: Correlation of individual measurements of PSMA-binder uptake versus individually measured pain sensitivity for CFA-treated inflammatory pain animals. The correlation factor R shows a very robust correlation between these two values. This shows, that indeed, not only the location but also the degree of inflammation induced pain sensitivity can be measured by PSMA-binders.

FIG. 8: Correlation of individual measurements of PSMA-binder uptake versus individually measured pain sensitivity for SNI-treated neuropathic pain animals. Left graph correlates the data taken from the site of lesion. Right graph correlates the data taken from the nerve plexus. Both but especially the nerve plexus values show strong correlation between binder uptake and pain sensitivity showing that PSMA-binder uptake is a good correlate of neuropathic pain measurement.

FIG. 9 A) to C): Results of CT, PET and CT/PET analysis of a patient with lower back pain using PSMA ligands. Painful areas in the lower back correlate with the signal intensity of PSMA-ligand.

FIG. 10: Comparative data obtain in control patients and patients with fibromyalgia

FIG. 11: Pain sensitivity is correlated to [¹⁸F]DCFPyl uptake in the “spared nerve injury (SNI)” mouse model. A: Pain sensitivity of the affected paw (measured with the Dynamic Plantar Test) is significantly increased relative to naive animals 7 and 14 days after nerve ligation. B: Tracer uptake at the lesion site is significantly increased 3, 7, and 14 days after surgery. C: Pain sensitivity and tracer uptake are significantly correlated. D: Examples of PET images from a sham animal (nerve was exposed by surgery but not ligated) and an SNI animal after 7 days. Arrows indicate lesion site.

FIG. 12: Pain sensitivity is correlated to [¹⁸F]DCFPyl uptake in the inflammatory “Complete Freunds Adjuvant (CFA)” mouse model. A: Pain sensitivity of the affected paw (measured with the Dynamic Plantar Test) is significantly increased relative to naive animals 2, 7 and 14 days after CFA injection. B: Tracer uptake at the lesion site is significantly increased 2, 7, and 14 days after injection. C: Pain sensitivity and tracer uptake are significantly correlated. D: PET image from a CFA animal after 2 days. Arrows indicate lesion site.

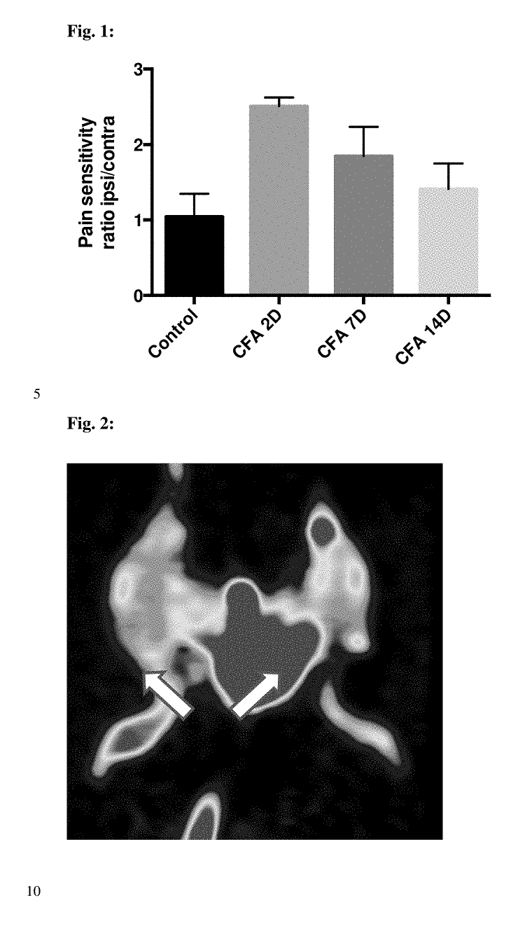

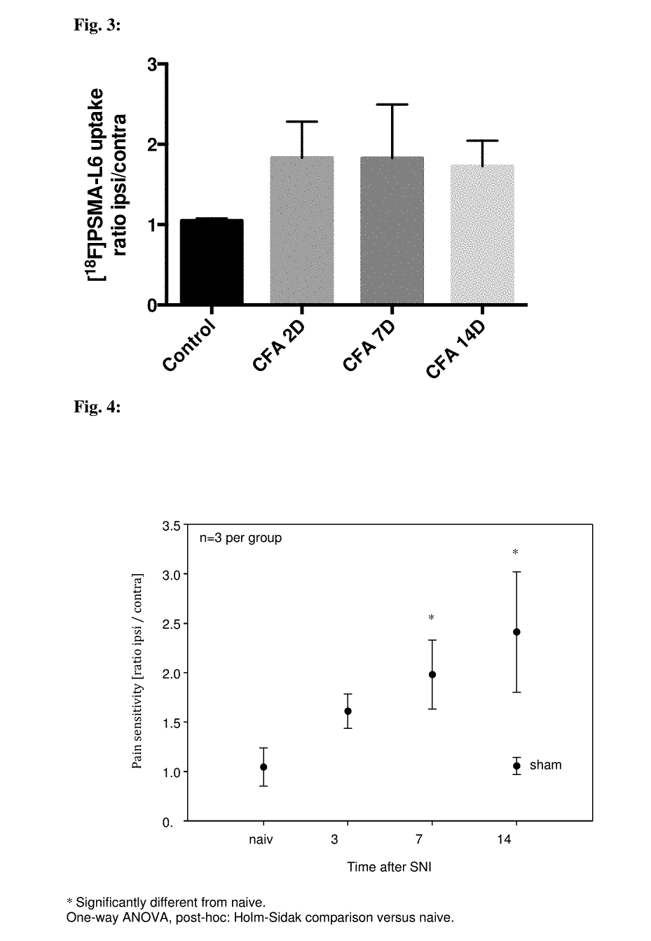

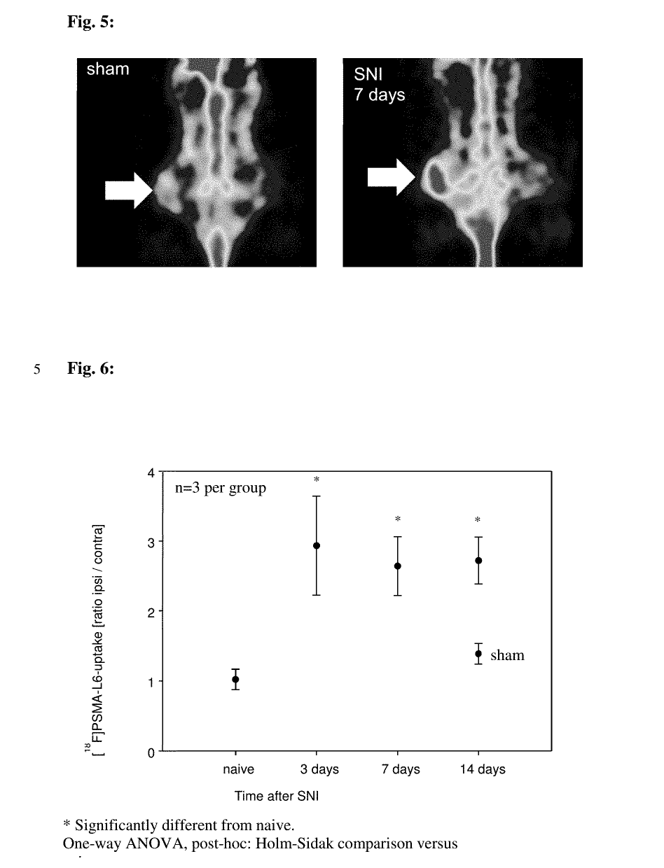

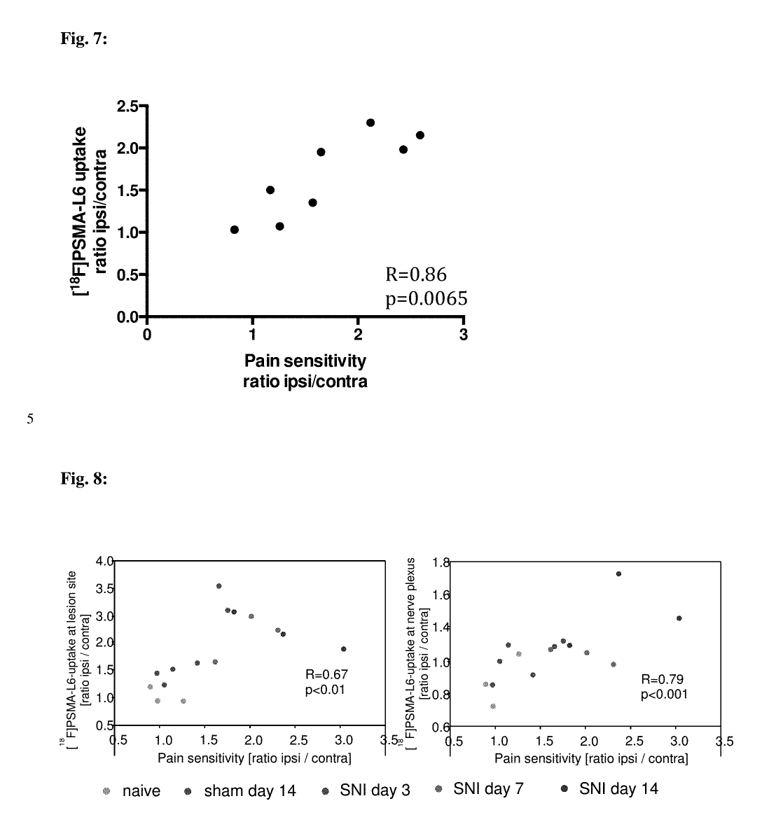

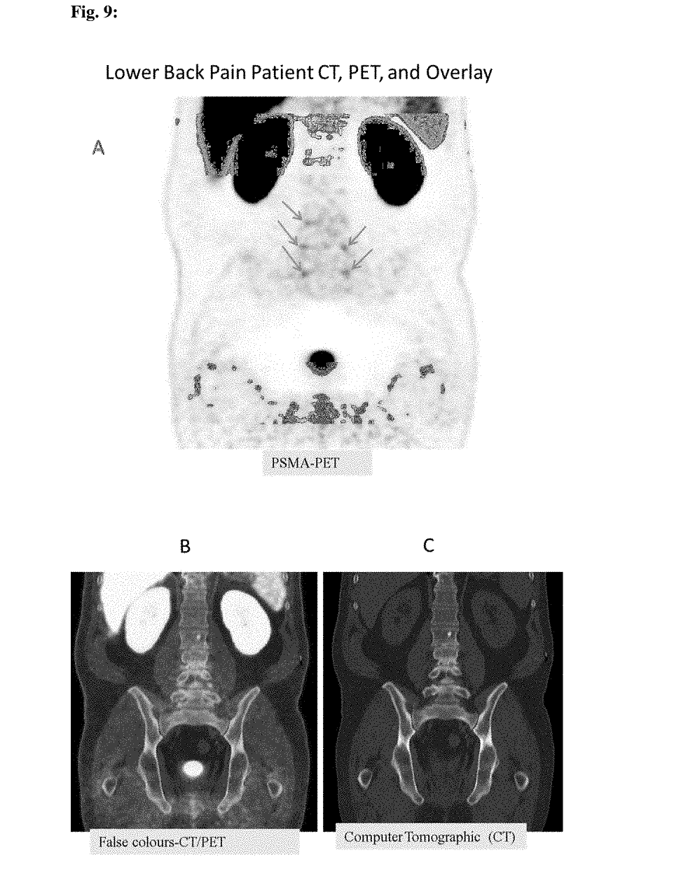

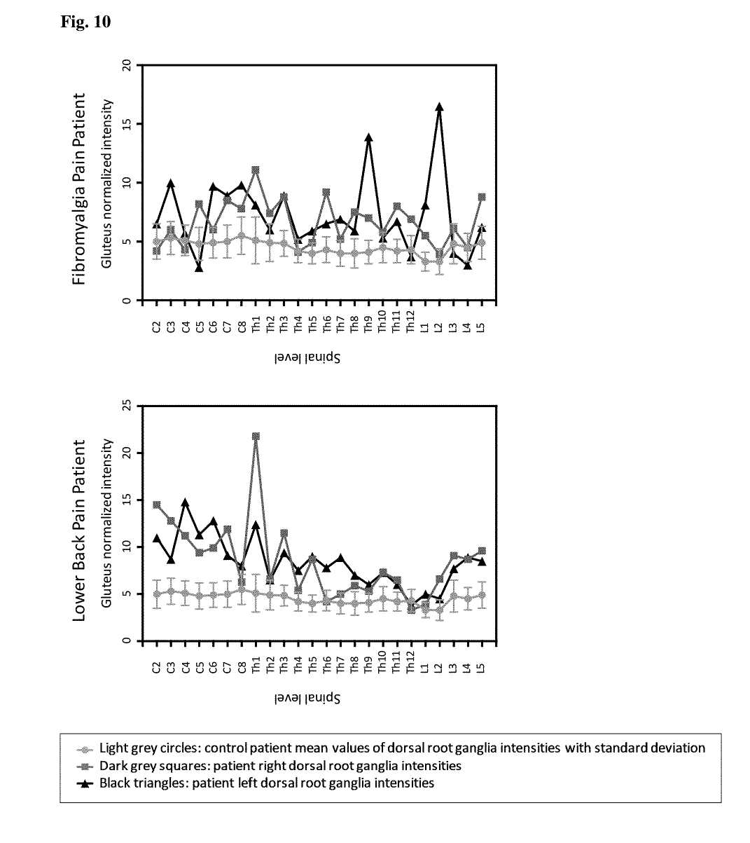

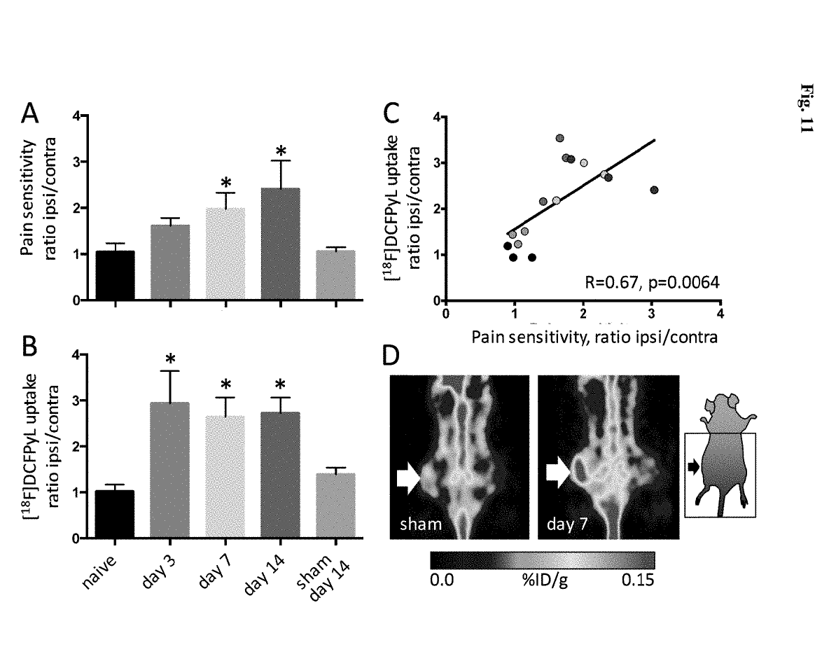

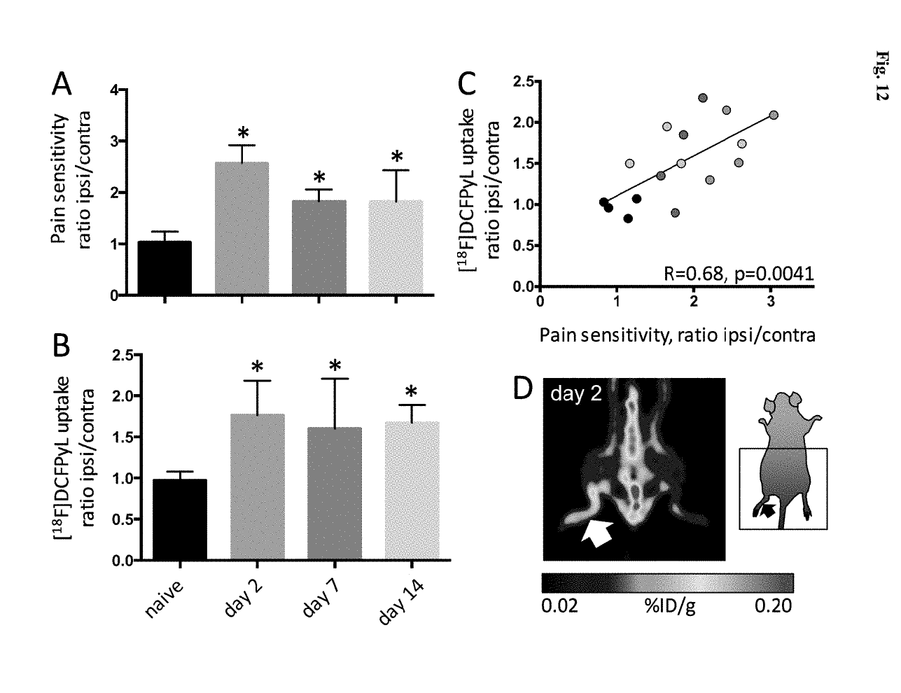

### REFERENCE LIST

Andlin-Sobocki P, Jonsson B, Wittchen H U, Olesen J (2005) Cost of disorders of the brain in Europe. European journal of neurology: the official journal of the European Federation of Neurological Societies 12 Suppl 1:1-27.
Barinka C, Rojas C, Slusher B, Pomper M (2012) Glutamate carboxypeptidase II in diagnosis and treatment of neurologic disorders and prostate cancer. Current medicinal chemistry 19:856-870.
Barton J L, Criswell L A, Kaiser R, Chen Y H, Schillinger D (2009) Systematic review and metaanalysis of patient self-report versus trained assessor joint counts in rheumatoid arthritis. J Rheumatol 36:2635-2641.
Berger U V, Carter R E, McKee M, Coyle J T (1995) N-acetylated alpha-linked acidic dipeptidase is expressed by non-myelinating Schwann cells in the peripheral nervous system. J Neurocytol 24:99-109.
Bostwick D G, Pacelli A, Blute M, Roche P, Murphy G P (1998) Prostate specific membrane antigen expression in prostatic intraepithelial neoplasia and adenocarcinoma: a study of 184 cases. Cancer 82:2256-2261.
Bourquin A F, Suveges M, Pertin M, Gilliard N, Sardy S, Davison A C, Spahn D R, Decosterd I (2006) Assessment and analysis of mechanical allodynia-like behavior induced by spared nerve injury (SNI) in the mouse. Pain 122:14 ell-14.
Brown D G, Krupp J J (2006)N-methyl-D-aspartate receptor (NMDA) antagonists as potential pain therapeutics. Curr Top Med Chem 6:749-770.
Carlton S M, Hargett G L, Coggeshall R E (1995) Localization and activation of glutamate receptors in unmyelinated axons of rat glabrous skin. Neurosci Lett 197:25-28.
Carlton S M, Hargett G L, Coggeshall R E (2001) Localization of metabotropic glutamate receptors 2/3 on primary afferent axons in the rat. Neuroscience 105:957-969.
Chang S S, Reuter V E, Heston W D, Bander N H, Grauer L S, Gaudin P B (1999) Five different anti-prostate-specific membrane antigen (PSMA) antibodies confirm PSMA expression in tumor-associated neovasculature. Cancer Res 59:3192-3198.
Chen Y, Pullambhatla M, Foss C A, Byun Y, Nimmagadda S, Senthamizhchelvan S, Sgouros G, Mease R C, Pomper M G (2011) 2-(3-{1-Carboxy-5-[(6-[18F]fluoro-pyridine-3-carbonyl)-amino]-pentyl}-ureido)-pen tanedioic acid, [18F]DCFPyL, a PSMA-based PET imaging agent for prostate cancer. Clin Cancer Res 17:7645-7653.
Coderre T J, Kumar N, Lefebvre C D, Yu J S (2007) A comparison of the glutamate release inhibition and anti-allodynic effects of gabapentin, lamotrigine, and riluzole in a model of neuropathic pain. J Neurochem 100:1289-1299.
Collins S, Sigtermans M J, Dahan A, Zuurmond W W, Perez R S (2010) NMDA receptor antagonists for the treatment of neuropathic pain. Pain Med 11:1726-1742.
Davidson E M, Coggeshall R E, Carlton S M (1997) Peripheral NMDA and non-NMDA glutamate receptors contribute to nociceptive behaviors in the rat formalin test. Neuroreport 8:941-946.
Decosterd I, Woolf C J (2000) Spared nerve injury: an animal model of persistent peripheral neuropathic pain. Pain 87:149-158.
deGroot J, Zhou S, Carlton S M (2000) Peripheral glutamate release in the hindpaw following low and high intensity sciatic stimulation. Neuroreport 11:497-502.
Doble A (1999) The role of excitotoxicity in neurodegenerative disease: implications for therapy. Pharmacol Ther 81:163-221.
Fillingim R B et al. (2014) The ACTTION-American Pain Society Pain Taxonomy (AAPT): an evidence-based and multidimensional approach to classifying chronic pain conditions. J Pain 15:241-249.
Gala J L, Loric S, Guiot Y, Denmeade S R, Gady A, Brasseur F, Heusterspreute M, Eschwege P, De Nayer P, Van Cangh P, Tombal B (2000) Expression of prostate-specific membrane antigen in transitional cell carcinoma of the bladder: prognostic value? Clin Cancer Res 6:4049-4054.
Greve K W, Bianchini K J, Brewer S T (2013) The assessment of performance and self-report validity in persons claiming pain-related disability. The Clinical neuropsychologist 27:108-137.
Haffner M C, Kronberger I E, Ross J S, Sheehan C E, Zitt M, Muhlmann G, Ofner D, Zelger B, Ensinger C, Yang X J, Geley S, Margreiter R, Bander N H (2009) Prostate-specific membrane antigen expression in the neovasculature of gastric and colorectal cancers. Human pathology 40:1754-1761.
Herr K, Coyne P J, McCaffery M, Manworren R, Merkel S (2011) Pain assessment in the patient unable to self-report: position statement with clinical practice recommendations. Pain management nursing: official journal of the American Society of Pain Management Nurses 12:230-250.
Imre G (2007) The preclinical properties of a novel group II metabotropic glutamate receptor agonist LY379268. CNS drug reviews 13:444-464.
Jackson D L, Graff C B, Richardson J D, Hargreaves K M (1995) Glutamate participates in the peripheral modulation of thermal hyperalgesia in rats. Eur J Pharmacol 284:321-325.
Kinoshita Y, Kuratsukuri K, Landas S, Imaida K, Rovito P M, Jr., Wang C Y, Haas G P (2006) Expression of prostate-specific membrane antigen in normal and malignant human tissues. World journal of surgery 30:628-636.
Lau A, Tymianski M (2010) Glutamate receptors, neurotoxicity and neurodegeneration. Pflugers Arch 460:525-542.
Lawand N B, Willis W D, Westlund K N (1997) Excitatory amino acid receptor involvement in peripheral nociceptive transmission in rats. Eur J Pharmacol 324:169-177.
Li D, Puntillo K, Miaskowski C (2008) A review of objective pain measures for use with critical care adult patients unable to self-report. J Pain 9:2-10.
Lopes A D, Davis W L, Rosenstraus M J, Uveges A J, Gilman S C (1990) Immunohistochemical and pharmacokinetic characterization of the site-specific immunoconjugate CYT-356 derived from antiprostate monoclonal antibody 7E11-C5. Cancer Res 50:6423-6429.
Luthi-Carter R, Barczak A K, Speno H, Coyle J T (1998) Hydrolysis of the neuropeptide N-acetylaspartylglutamate (NAAG) by cloned human glutamate carboxypeptidase II. Brain Res 795:341-348.
Marko J, Gould C F, Bonavia G H, Wolfman D J (2015) State-of-the-art imaging of prostate cancer. Urologic oncology.
McNearney T, Speegle D, Lawand N, Lisse J, Westlund K N (2000) Excitatory amino acid profiles of synovial fluid from patients with arthritis. J Rheumatol 27:739-745.
Mhawech-Fauceglia P, Zhang S, Terracciano L, Sauter G, Chadhuri A, Herrmann F R, Penetrante R (2007) Prostate-specific membrane antigen (PSMA) protein expression in normal and neoplastic tissues and its sensitivity and specificity in prostate adenocarcinoma: an immunohistochemical study using multiple tumour tissue microarray technique. Histopathology 50:472-483.
Montana M C, Gereau R W (2011) Metabotropic glutamate receptors as targets for analgesia: antagonism, activation, and allosteric modulation. Current pharmaceutical biotechnology 12:1681-1688.
Montana M C, Cavallone L F, Stubbert K K, Stefanescu A D, Kharasch E D, Gereau RWt (2009) The metabotropic glutamate receptor subtype 5 antagonist fenobam is analgesic and has improved in vivo selectivity compared with the prototypical antagonist 2-methyl-6-(phenylethynyl)-pyridine. J Pharmacol Exp Ther 330:834-843.
Montana M C, Conrardy B A, Cavallone L F, Kolber B J, Rao L K, Greco S C, Gereau RWt (2011) Metabotropic glutamate receptor 5 antagonism with fenobam: examination of analgesic tolerance and side effect profile in mice. Anesthesiology 115:1239-1250.
Niswender C M, Conn P J (2010) Metabotropic glutamate receptors: physiology, pharmacology, and disease. Annu Rev Pharmacol Toxicol 50:295-322.
Omote K, Kawamata T, Kawamata M, Namiki A (1998) Formalin-induced release of excitatory amino acids in the skin of the rat hindpaw. Brain Res 787:161-164.
Pinto J T, Suffoletto B P, Berzin T M, Qiao C H, Lin S, Tong W P, May F, Mukherjee B, Heston W D (1996) Prostate-specific membrane antigen: a novel folate hydrolase in human prostatic carcinoma cells. Clin Cancer Res 2:1445-1451.
Qi J, Leahy R M, Cherry S R, Chatziioannou A, Farquhar T H (1998) High-resolution 3D Bayesian image reconstruction using the microPET small-animal scanner. Physics in medicine and biology 43:1001-1013.
Renfrey S, Downton C, Featherstone J (2003) The painful reality. Nat Rev Drug Discov 2:175-176.
Robinson M B, Blakely R D, Couto R, Coyle J T (1987) Hydrolysis of the brain dipeptide N-acetyl-L-aspartyl-L-glutamate. Identification and characterization of a novel N-acetylated alpha-linked acidic dipeptidase activity from rat brain. J Biol Chem 262:14498-14506.
Rovenska M, Hlouchova K, Sacha P, Mlcochova P, Horak V, Zamecnik J, Barinka C, Konvalinka J (2008) Tissue expression and enzymologic characterization of human prostate specific membrane antigen and its rat and pig orthologs. Prostate 68:171-182.
Sacha P, Zamecnik J, Barinka C, Hlouchova K, Vicha A, Mlcochova P, Hilgert I, Eckschlager T, Konvalinka J (2007) Expression of glutamate carboxypeptidase II in human brain. Neuroscience 144:1361-1372.
Schmidtko A, Gao W, Konig P, Heine S, Motterlini R, Ruth P, Schlossmann J, Koesling D, Niederberger E, Tegeder I, Friebe A, Geisslinger G (2008a) cGMP produced by NO-sensitive guanylyl cyclase essentially contributes to inflammatory and neuropathic pain by using targets different from cGMP-dependent protein kinase I. J Neurosci 28:8568-8576.
Schmidtko A, Gao W, Sausbier M, Rauhmeier I, Sausbier U, Niederberger E, Scholich K, Huber A, Neuhuber W, Allescher H D, Hofmann F, Tegeder I, Ruth P, Geisslinger G (2008b) Cysteine-rich protein 2, a novel downstream effector of cGMP/cGMP-dependent protein kinase I-mediated persistent inflammatory pain. J Neurosci 28:1320-1330.
Silver D A, Pellicer I, Fair W R, Heston W D, Cordon-Cardo C (1997) Prostate-specific membrane antigen expression in normal and malignant human tissues. Clin Cancer Res 3:81-85.
Sokoloff R L, Norton K C, Gasior C L, Marker K M, Grauer L S (2000) A dual-monoclonal sandwich assay for prostate-specific membrane antigen: levels in tissues, seminal fluid and urine. Prostate 43:150-157.
Srinivasarao M, Galliford C V, Low P S (2015) Principles in the design of ligand-targeted cancer therapeutics and imaging agents. Nat Rev Drug Discov 14:203-219.
Stewart W F, Ricci J A, Chee E, Morganstein D, Lipton R (2003) Lost productive time and cost due to common pain conditions in the US workforce. JAMA 290:2443-2454.
Troyer J K, Beckett M L, Wright G L, Jr. (1995) Detection and characterization of the prostate-specific membrane antigen (PSMA) in tissue extracts and body fluids. Int J Cancer 62:552-558.
Wang W, Tavora F, Sharma R, Eisenberger M, Netto G J (2009) PSMA expression in Schwannoma: a potential clinical mimicker of metastatic prostate carcinoma. Urologic oncology 27:525-528.
Woodcock J (2009) A difficult balance—pain management, drug safety, and the FDA. N Engl J Med 361:2105-2107.
Wozniak K M, Rojas C, Wu Y, Slusher B S (2012) The role of glutamate signaling in pain processes and its regulation by GCP II inhibition. Current medicinal chemistry 19:1323-1334.
Zammataro M, Chiechio S, Montana M C, Traficante A, Copani A, Nicoletti F, Gereau RWt (2011) mGlu2 metabotropic glutamate receptors restrain inflammatory pain and mediate the analgesic activity of dual mGlu2/mGlu3 receptor agonists. Mol Pain 7:6.
Zhou S, Bonasera L, Carlton S M (1996) Peripheral administration of NMDA, AMPA or K A results in pain behaviors in rats. Neuroreport 7:895-900.

1. A PSMA-binding molecule comprising a detectable unit for use in the diagnosis and/or imaging of pain in a patient suffering from pain or in a patient that is suspected to suffer from pain.

2. The PSMA-binding molecule comprising a detectable unit for use in the diagnosis and/or imaging of pain, wherein said patient suspected to suffer from pain is unable to communicate verbally.

3. The PSMA-binding molecule comprising a detectable unit according to claim 1, wherein the detectable unit has a structure depicted in formula I 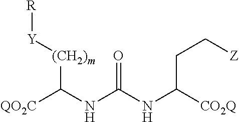
wherein
Z is tetrazole or CO₂Q;
each Q is hydrogen; and
wherein
(A) m is 0, 1, 2, 3, 4, 5, or 6;
R is a pyridine ring selected from the group consisting of 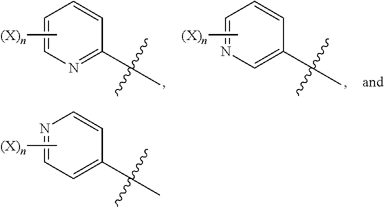
wherein X is a radioisotope of fluorine, a radioisotope of iodine, a radioisotope of bromine, a radioisotope of astatine, —NHN═CHR³, CH₂R³;
n is 1, 2, 3, 4, or 5;
Y is O, S, N(R′), C(O), NR′C(O), C(O)N(R′), OC(O), C(O)O, NR′C(O)NR′, NR′C(S)NR′, NR'S(O)₂, S(CH₂)ₚ, NR′(CH₂)ₚ, O(CH₂)ₚ, OC(O)CHR⁸NHC(O), NHC(O)CHR⁸NHC(O), or a covalent bond; wherein p is 1, 2, or 3, R′ is H or C₁-C₆ alkyl, and R⁸ is hydrogen, alkyl, aryl or heteroaryl, each of which may be substituted;
R³ is alkyl, alkenyl, alkynyl, aryl, or heteroaryl each of which is substituted by a radioisotope of fluorine, a radioisotope of iodine, a radioisotope of bromine, or a radioisotope of astatine.

4. The PSMA-binding molecule comprising a detectable unit according to claim 1 for use in the diagnosis and/or imaging of pain in a patient suffering from pain or in a patient that is suspected to suffer from pain, comprising a structure depicted in formula II:
A-(B)_{b}-C (II);
wherein A is a metal chelator; suitable chelators consist of but not limited to DOTA, NOTA, DTPA, cDTPA, CHX-A″-DTPA, TETA, NODAGA, HBED, DFO, DOTAGA; PCTA, MA-NOTMP; TRAP-Pr, NOPO; DOTPI, H₄OCTAPA; DOTAGA; LI-1,2HOPO; H₂dedPA, AAZTA, DATA^{x}; B is a linker; C is a PSMA-binding molecule; and b is 1-5.

5. The PSMA-binding molecule comprising a detectable unit according to claim 4 for use in the diagnosis and/or imaging of pain in a patient suffering from pain or in a patient that is suspected to suffer from pain, wherein said molecule is selected from the group comprising compounds of formulae (III) to (XI): 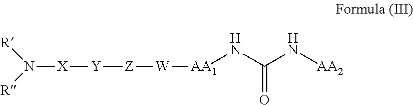
wherein
AA₁ and AA₂ each independently a natural or unnatural amino acid;
R′ is —CO—NR^{x}R^{y}—, —CS^{x}R^{y}—, COR^{x}, CSR^{x}, C(NR^{x})R^{x}, —S(O)ₚR^{x}—, —CO₂—NR^{x}R^{y}—, or optionally substituted alkyl;
R^{x} is optionally substituted aryl or optionally substituted alkyl;
R^{y} is H, optionally substituted aryl or optionally substituted alkyl;
X and Z are each independently C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, or a bond, each of which may be substituted with 0-5 R_{A};
Y and W are each independently —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl; 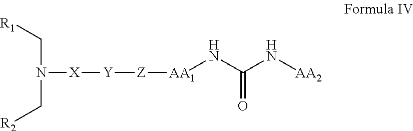
wherein
R₁ and R₂ are each independently selected from optionally substituted aryl, optionally substituted heteroaryl, optionally substituted heterocyclo, —COOH, hydroxyl, optionally substituted alkoxy, amino, optionally substituted mono or dialkylamino, thiol, and optionally substituted alkylthiol;
AA₁ and AA₂ are each independently a natural or unnatural amino acid;
X and Z are each independently C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, or a bond, each of which may be substituted with 0-5 R_{A};
Y is —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl; 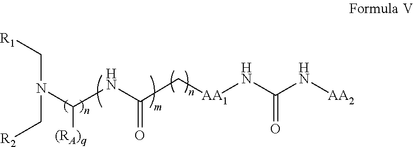
wherein
AA₁ and AA₂ are each independently a natural amino acid;
R₁ is pyridyl, pyrimidinyl, pyrazinyl, pyridizinyl, quinolinyl, thienyl, thiazolyl, oxazolyl, isoxazolyl, pyrrolyl, furanyl, isoquinolinyl, imiazolyl, or triazolyl;
R₂ is pyridyl, pyrimidinyl, pyrazinyl, pyridizinyl, quinolinyl, thienyl, thiazolyl, oxazolyl, isoxazolyl, pyrrolyl, furanyl, isoquinolinyl, or triazolyl, —COOH, hydroxyl, alkoxy, amino, mono or dialkylamino;
R_{A}, for each occurrence, is halogen, hydroxy, amino, cyano, nitro, or CO₂H;
m is 0 or 1;
each n is independently 1-8; and
each q is independently 0 or 1; 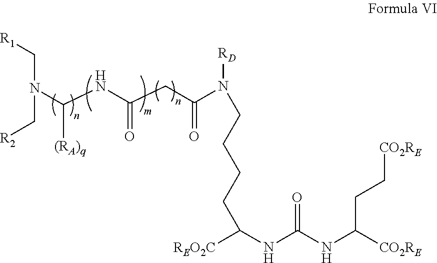
wherein
each R_{D} is independently H, optionally substituted alkyl, optionally substituted cycloalkyl, optionally substituted aryl, optionally substituted heteroaryl, optionally substituted heterocyclo, or optionally substituted aralkyl;
each R_{E} is independently H, optionally substituted alkyl, optionally substituted cycloalkyl, optionally substituted aryl, optionally substituted heteroaryl, optionally substituted heterocyclo, or optionally substituted aralkyl;
R₁ is pyridyl, pyrimidinyl, pyrazinyl, pyridizinyl, isoquinolinyl, imiazolyl, or quinolinyl;
R₂ is pyridyl, pyrimidinyl, pyrazinyl, pyridizinyl, isoquinolinyl, quinolinyl; —COOH, hydroxyl, alkoxy, amino, mono or dialkylamino;
R_{A}, for each occurrence, is hydroxy, amino, or CO₂H;
each m is independently 0 or 1; and
each n is independently 1-8; 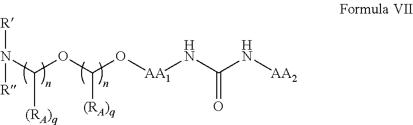
wherein
AA₁ and AA₂ are each independently a natural amino acid;
R′ is —CO—NR^{x}R^{y}—, —CS—NR^{x}R^{y}—, COR^{x}, CSR^{x}, C(NR^{x})R^{x}, —S(O)ₚR^{x}—, —CO₂—NR^{x}R^{y}—, or optionally substituted alkyl;
R″ is H or optionally substituted alkyl;
R″ is optionally substituted aryl or optionally substituted alkyl;
R^{y} is H, optionally substituted aryl or optionally substituted alkyl;
R_{A}, for each occurrence, is halogen, hydroxy, amino, cyano, nitro, or CO₂H;
each n is independently 0-8; and
each q is independently 0 or 1; 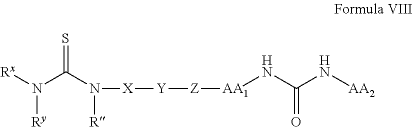
wherein
R″ is H or optionally substituted alkyl;
R^{x} is optionally substituted aryl or optionally substituted alkyl;
R^{y} is H, optionally substituted aryl or optionally substituted alkyl;
AA₁ and AA₂ are each independently a natural or unnatural amino acid;
X and Z are each independently C₁-C₈ alkyl, C₂-C₈ alkenyl, or C₂-C₈ alkynyl, C₁-C₈ heteroalkyl, C₂-C₈ heteroalkenyl, or C₂-C₈ heteroalkynyl, C₁-C₈alkoxy, or a bond, each of which may be substituted with 0-5 R_{A};
Y is —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl; 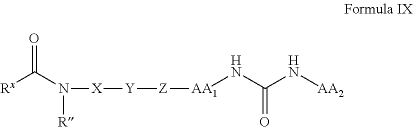
wherein
R″ is H or optionally substituted alkyl;
R^{x} is optionally substituted aryl or optionally substituted alkyl;
AA₁ and AA₂ are each independently a natural or unnatural amino acid;
X and Z are each independently C₁-C₈ alkyl, C₂-C₈ alkenyl, or C₂-C₈ alkynyl, C₁-C₈ heteroalkyl, C₂-C₈ heteroalkenyl, or C₂-C₈ heteroalkynyl, C₁-C₈alkoxy, or a bond, each of which may be substituted with 0-5 R_{A};
Y is —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl; 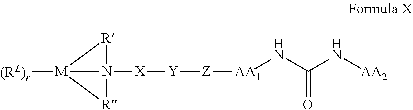
wherein
M is a metal or Al—F;
R^{L} is a metal ligand;
R′ is —CO—NR^{x}R^{y}—, —CS—NR^{x}R^{y}—, COR^{x}, CSR^{x}, C(NR^{x})R^{x}, —S(O)ₚR^{x}—, —CO₂—NR^{x}R^{y}—, or optionally substituted alkyl;
R″ is H or optionally substituted alkyl;
R^{x} is optionally substituted aryl or optionally substituted alkyl;
R^{y} is H, optionally substituted aryl or optionally substituted alkyl;
X and Z are each independently C₁-C₈alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, or a bond, each of which may be substituted with 0-5 R_{A};
Y and W are each independently —O—, —S(O)ₚ—, —NH—, —NR_{B}—, —CH═CH—, —CR_{B}═CH—, —CH═CR_{B}—, —NH—CO—, —NH—CO₂—, —NR_{B}—CO—, —NR_{B}—CO₂—; —CO—NH—, —CO₂—NH—, —CO—NR_{B}—, —CO₂—NR_{B}—, or a bond;
p is 0, 1, or 2;
R_{A}, for each occurrence, is halogen, hydroxy, amino, cyano, nitro, CO₂H, optionally substituted alkyl, optionally substituted cycloalkyl, optionally substituted heterocyclo, optionally substituted alkenyl, optionally substituted alkynyl, optionally substituted alkoxy, optionally substituted mono or dialkylamino, optionally substituted alkylthio, optionally substituted alkylsulfinyl, optionally substituted alkylsulfonyl, optionally substituted mono- or dialkylcarboxamide, optionally substituted aryl, or optionally substituted heteroaryl; and
R_{B}, for each occurrence, is optionally substituted alkyl, optionally substituted alkoxy, optionally substituted mono or dialkylamino, optionally substituted alkylthio, optionally substituted aryl, or optionally substituted heteroaryl and
r is 1-5; and 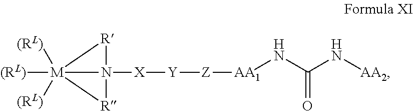
wherein the definitions of the residues are the same as in Formula X.

6. The PSMA-binding molecule for use in diagnosis and/or imaging of pain in a subject suffering from pain or in a patient that is suspected to suffer from pain according to claim 1, having the structure

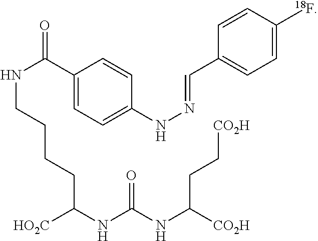

7. The PSMA-binding molecule for use in diagnosis and/or imaging of pain in a subject suffering from pain or in a patient that is suspected to suffer from pain according to claim 1, having the structure

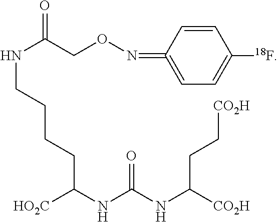

8. The PSMA-binding molecule as defined in claim 1 for use in diagnosis or imaging of pain, wherein the pain eliciting location is visualized.

9. The PSMA-binding molecule as defined in claim 1 for use in diagnosis or imaging of pain in a subject suffering from pain or in a patient that is suspected to suffer from pain, wherein the level of enzyme PSMA is increased at a site of pain along a peripheral nerve or parts thereof.

10. The PSMA-binding molecule for use in diagnosis or imaging of pain in a subject suffering from pain or in a patient that is suspected to suffer from pain according to claim 9, wherein the increased level of enzyme PSMA at said site of pain is detected as intensity of said tracer compound I after administration to said subject and wherein said tracer compound intensity at the site of pain is statistically increased in comparison to a) said tracer compound intensity at the site of an unaffected contralateral site and/or b) to a threshold that has been statistically determined.

11. The PSMA-binding molecule for use in diagnosis or imaging of pain in in a subject suffering from pain or in a patient that is suspected to suffer from pain according to claim 1, wherein diagnosis or imaging of pain may be the visualization of the pain eliciting location, the determination of pain sensitivity, and/or the determination of the aetiology of pain.

12. The PSMA-binding molecule for use in diagnosis or imaging of pain in a subject suffering from pain according or in a patient that is suspected to suffer from pain to claim 1, wherein it is differentiated between peripherally caused pain (peripheral pain) versus central and periphery independent pain.

13. The PSMA-binding molecule for use in diagnosis or imaging of pain in a subject suffering from pain or in a patient that is suspected to suffer from pain according to claim 1, wherein it is determined whether said subject suffers from inflammatory pain or neuropathic pain.

14. The PSMA-binding molecule according to any one of the preceding claims in the manufacture of a kit for the diagnosis and/or imaging of pain in a patient suffering from pain according or in a patient that is suspected to suffer from pain to claim 1.

15. A kit comprising a container comprising PSMA-binding molecule as defined in claim 1, for the diagnosis and/or imaging of pain, optionally comprising instructions for use, and further optionally comprising information on the interpretation of imaging results.

16. A method for diagnosing or imaging of pain in a subject suffering from pain or in a patient that is suspected to suffer from pain comprising administering to said subject an effective amount of a compound according to claim 1.

17. An in vitro method of imaging cells, organs, tissue samples, wherein the cells, organs or tissue samples are exposed to a chemical or physical stimulus suspect to be involved in the development or reduction of pain, and the expression and/or quantity of PSMA is determined using a PSMA-binding molecule as defined in claim 1.

18. A method for diagnosing or imaging of pain in a subject suffering from pain or suspected to suffer from pain comprising subjecting a subject, to whom an effective amount of a compound according to claim 1 has been administered, to PET-imaging.
